(19) United States
(12) Patent Application Publication
(10) Pub. No.: US 2017/0128172 A1
(43) Pub. Date: May 11, 2017

(54) MATERIALS AND METHODS FOR FILLING DENTAL BONE VOIDS

(71) Applicants: 3-D MATRIX, LTD., Tokyo (JP); Forsyth Dental Infirmary for Children, Cambridge, MA (US)

(72) Inventors: Lisa Spirio, Lexington, MA (US); Hatice Hasturk, Brighton, MA (US); Thomas E. Van Dyke, West Roxbury, MA (US)

(21) Appl. No.: 15/318,568

(22) PCT Filed: Jun. 19, 2015
(86) PCT No.: PCT/US15/36590

§ 371 Date: Dec. 13, 2016

Related U.S. Application Data
(60) Provisional application No. 62/015,114, filed on Jun. 20, 2014.

(51) Int. Cl. A61C 8/00; A61C 19/06; A61L 27/38; A61L 27/22; A61L 27/10; A61L 27/54; A61C 8/02; A61L 27/52

(52) U.S. Cl. CPC A61C 8/0092; A61C 8/0006; A61C 19/063; A61L 27/52; A61L 27/227; A61L 27/10; A61L 27/54; A61L 27/3865; A61L 2430/12; A61L 2300/412; A61L 2400/12; A61L 2300/406; A61L 2300/41

(57) ABSTRACT

Materials and methods for dental bone void filling such as during a sinus lift procedure are provided. A peptide comprising between about 7 amino acids and about 32 amino acids in a solution may be introduced to a target site. The peptide may undergo self-organization under physiological conditions and/or in the presence of a cation.

### FIELD OF THE TECHNOLOGY

One or more aspects relate generally to materials and methods that may be used in medical, research, and industrial applications. More particularly, one or more aspects relate to materials and methods that may be used to fill dental bone voids, including membranes, hydrogels, compositions, and solutions that may be used to facilitate sinus lift procedures.

### BACKGROUND

Sinus elevation procedures have been amply described in the last twenty years as a successful method of ridge preservation by augmenting the posterior maxilla for future implant placement in cases of pneumatization of the maxillary sinuses.

Several materials for sinus bone grafts have been tested. A 90% success rate in a three to five year time frame was reported in a survival analysis of implants placed into augmented sinuses. This value is better than results published for implants placed in native maxillary bone with no bone graft used.

Based on the analysis of the literature, it is still not possible to state with certainty which material or technique is superior for sinus augmentation. One of the challenges related to sinus lift surgery is the long healing time required between grafting, implant placement, and restoration of the area. The use of autogenous bone as an augmentation material is generally considered to be superior but requires a second surgical site for collection. The use of allografts carries the potential risk of disease transmission.

### SUMMARY

In accordance with one or more aspects, a method of performing a sinus lift procedure on a subject is provided. The method may comprise introducing a delivery device into a mouth of the subject, positioning an end of the delivery device proximate a target site in a posterior maxilla of the subject where promotion of alveolar bone growth is desired, administering through the delivery device a solution comprising a self-assembling peptide comprising between about 7 and about 32 amino acids in an effective amount and in an effective concentration to form a hydrogel scaffold under physiological conditions to promote alveolar bone growth at the target site, and removing the delivery device from the mouth of the subject.

In accordance with one or more aspects, a kit for filling a dental bone void in a subject is provided. The kit may comprise a solution comprising a self-assembling peptide comprising between about 7 amino acids and about 32 amino acids in an effective amount and in an effective concentration to form a hydrogel scaffold under physiological conditions to promote alveolar bone growth at a target site, and instructions for administering the solution to the target site in an alveolar bone of the subject.

Still other aspects and embodiments are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. The accompanying drawings are included to provide illustration and a further understanding of the various aspects and embodiments, and are incorporated in and constitute a part of this specification. The drawings, together with the remainder of the specification, serve to explain principles and operations of the described and claimed aspects and embodiments.

### BRIEF DESCRIPTION OF THE DRAWINGS

The accompanying drawings are not intended to be drawn to scale. For purposes of clarity, not every component may be labeled. In the drawings which are referenced in the accompanying Example:

FIG. 1 is a schematic timeline of the study protocol;

FIG. 2A is a photograph of a lateral sinus wall augmentation with PuraMatrix®. A window in the lateral wall is shown, in accordance with some embodiments;

FIG. 2B is a photograph of a lateral sinus wall augmentation with PuraMatrix®. The site filled with PuraMatrix® is shown, in accordance with some embodiments;

FIG. 2C is a photograph of a lateral sinus wall augmentation with PuraMatrix®. The site is closed with CollaTape® (Integra Lifesciences Corporation), in accordance with some embodiments;

FIG. 3A is an image of a crestal zone of a representative specimen at 100× magnification which had 6 mm of residual crest prior to grafting with DFDBA;

FIG. 3B is an image of a crestal zone of a representative specimen at 100× magnification which had 5.3 mm of residual crest prior to grafting with PuraMatrix®;

FIG. 4A is a graph of vital bone of graft area at three representative zones (crestal, crest+graft, and grafted) for DFDBA and PuraMatrix®;

FIG. 4B is a graph of total vital bone for DFDBA and PuraMatrix®;

FIG. 5A is a graph of percent bone marrow space at three representative zones (crestal, crest+graft, and grafted) for DFDBA and PuraMatrix®;

FIG. 5B is a graph of percent total bone marrow space for DFDBA and PuraMatrix® FIG. 6 is an image of a PuraMatrix® grafted area, in accordance with some embodiments;

FIG. 7 is an image of a PuraMatrix® grafted area, in accordance with some embodiments;

FIG. 8 is an image of a DFDBA grafted area, in accordance with some embodiments;

FIG. 9 is an image of a DFDBA grafted area, in accordance with some embodiments;

FIG. 10 is an image of a PuraMatrix® grafted area, in accordance with some embodiments;

FIG. 11 is an image of a PuraMatrix® grafted area, in accordance with some embodiments;

FIG. 12 is an image of a DFDBA grafted area, in accordance with some embodiments;

FIG. 13 is an image in which alveolar bone height and alveolar bone width was measured;

FIG. 14 is a bar graph comparing changes in bone height in subjects assigned to the PuraMatrix® group;

FIG. 15 is a bar graph comparing changes in bone height in subjects assigned to the DFDBA/Control group;

FIG. 16 is a graph comparing bone height change between PuraMatrix® and DFDBA groups, in accordance with some embodiments; and

FIG. 17 is a graph comparing bone height change between PuraMatrix® and DFDBA groups, in accordance with some embodiments.

### DETAILED DESCRIPTION

In accordance with one or more embodiments, materials and methods of the present disclosure may be used to fill dental bone voids such as those associated with a sinus lift procedure. Beneficially, the disclosed materials and methods may be associated with greater mechanical strength of implants, higher levels of biocompatibility, and more vital bone growth in comparison to conventional techniques.

In accordance with one or more specific embodiments, a peptide hydrogel may be used as a bone void filler (BVF) that resorbs and is replaced with bone during a healing process following administration at a target site. The peptide hydrogel may be placed into bony voids or gaps of the skeletal system, such as during a sinus lift procedure. In certain embodiments, self-assembling peptides and self-assembled structures thereof may be used as cell culture supports for the repair and replacement of various tissues and as a scaffold to encapsulate living cells. The peptide hydrogel may promote periodontal tissue regeneration and the production of related extracellular matrix proteins. In at least some embodiments, the peptide hydrogel is non-immunogenic and represents an improvement over existing materials for this indication, including demineralized freeze-dried bone allograft (DFDBA) preparations.

The materials and methods may find particular application in filling dental bone voids in a subject. As used herein, the term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the invention includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others.

In at least some embodiments, a subject candidate for a sinus lift procedure may generally have less than or equal to about 8 mm of residual vertical bone, as well as sufficient buccal and lingual bone width.

The filling of a dental bone void may be partial or complete. In at least some embodiments, vital bone density may be increased at a target site. In some embodiments, dental bone of a subject at a target site may be restored in part or in full. In various embodiments, a target site may be prepared such that an implant may be secured at the target site. Dental bone content may be augmented at a target site in accordance with one or more embodiments.

In at least some embodiments, a level of bone augmentation associated with a successful sinus lift procedure may be expected to provide an adequate bone width and depth to place an implant, such as a 3.3, 4.1 or 4.8 mm ITI implant.

In accordance with one or more embodiments, a target site may generally be any area or region in which promotion of alveolar bone growth is desired. In some embodiments, the target site may generally be associated with a surgical procedure. The target site may be located in any region of an alveolar bone of a subject, such as where an implant is desired. In some embodiments, the target site may be in a posterior maxilla of the subject, commonly referred to as the upper jaw.

In at least some embodiments, the target site may be associated with a sinus lift or sinus augmentation procedure intended to increase the likelihood of a successful implant. Bone may generally be added to the upper jaw of the patient, such as in the vicinity of the molar teeth. The bone may be added between the upper jaw and the maxillary sinuses. Space for new bone may generally be created by lifting or otherwise moving the sinus membrane upward. A dental professional may first make an incision in the gum tissue at the target site. The tissue may then be raised to expose the alveolar bone. A void may be opened in the bone. The membrane lining the sinus proximate the void generally separates the sinus from the jaw. This membrane may be pushed up and away from the jaw to create a space or void for bone growth. This space may be the target area as discussed herein.

As discussed in greater detail below, the materials and methods may include the administration, application, or injection of a self-assembling peptide, or a solution comprising a self-assembling peptide, or a composition comprising a self-assembling peptide, to a predetermined or desired target area. In some embodiments, the solution comprising a self-assembling peptide may be introduced into the dental bone void above the jaw. Once the solution has been administered, the tissue may be closed such as surgically with stitches. A period of time, for example three to twelve months, may be allowed to elapse prior to implantation. This period of time may generally allow for a desired degree of bone growth and meshing in the dental bone void.

In accordance with one or more embodiments, peptide hydrogels may be used alone or in combination with one or more of autogenous bone, allografts, alloplasts, or xenografts. These combinations may generally increase the volume of graft material and may also improve overall performance. In at least some embodiments, methods may involve mixing the peptide solution with an autograft or an allograft prior to administration.

In accordance with one or more embodiments, a method of performing a sinus lift procedure on a subject may involve introducing a delivery device into a mouth of the subject. An end of the delivery device may be positioned proximate a target site in a posterior maxilla of the subject where promotion of alveolar bone growth is desired. A solution comprising a self-assembling peptide comprising between about 7 and about 32 amino acids may be administered to the target site in an effective amount and in an effective concentration to form a hydrogel scaffold under physiological conditions to promote alveolar bone growth at the target site. The delivery device may then be removed from the mouth of the subject.

In some embodiments, the concentration effective to promote alveolar bone growth comprises a concentration in a range of about 0.1 weight per volume (w/v) percent to about 3 w/v percent peptide. The administered volume may vary as discussed herein, for example, based on the dimensions of the target site and/or the desired degree of bone augmentation. In some non-limiting embodiments, the volume of the administered peptide solution is between about 1 mL and about 5 mL. In some specific embodiments, the administered peptide solution may be PuraMatrix® peptide hydrogel.

In some methods, an implant may be secured into augmented alveolar bone at the target site after a predetermined period of time. In accordance with one or more embodiments, a healing period ranging from a couple of months to a couple of years may be associated with a sinus lift procedure to establish adequate bone regeneration at a target site. In some specific embodiments, healing of two months to one year may be required. In some embodiments, the predetermined period of time is between about three and about six months. In at least some embodiments, about six months of healing may be required. In some embodiments, additional doses of the peptide solution may be administered at the target site during the predetermined time period, randomly, upon visualization, or at regular intervals. In some embodiments, a supplemental volume of the peptide solution may be administered at the target site concurrently with implantation.

In other methods, an implant may be secured at the target site in the posterior maxilla concurrently with initial administration of the peptide solution.

In accordance with one or more embodiments, a sinus membrane may be supported in a region of the target site to provide a space for formation of the hydrogel scaffold. In some embodiments, this may involve the insertion and placement of a bather, such as a rigid barrier.

After administration, the target site may be surgically closed. A wound dressing may then be applied at the target site after administration of the peptide solution to facilitate healing and to help hold the peptide solution in place. The target site may be visualized after administration, such as at regular time intervals or after a predetermined period of time to assess alveolar bone augmentation.

In at least some embodiments, a self-assembled hydrogel scaffold at the target site may involve nanofibers having a diameter of about 10 nanometers to about 20 nanometers.

In accordance with one or more embodiments, the administered peptide solution is substantially non-biologically active. Sinus lift procedures in accordance with one or more embodiments may be associated with less than about 2 mm of radiographic bone loss at the target site upon implantation. The disclosed methods may be associated with no IgG reaction. The resulting augmented alveolar bone may be characterized by a vital bone density of at least about 35% in some non-limiting embodiments.

In accordance with one or more embodiments, routine adverse events associated with a sinus lift procedure may be experienced. For example, a sinus membrane perforation may occur during a grafting procedure. These may be treated based on standard of care.

Perforations may be covered with a collagen membrane and then the graft procedure may be resumed.

The term “self-assembling peptide” may refer to a peptide that may exhibit a beta-sheet structure in aqueous solution in the presence of specific conditions to induce the beta-sheet structure. These specific conditions may include increasing the pH of a self-assembling peptide solution. The increase in pH may be an increase in pH to a physiological pH. The specific conditions may also include adding a cation, such as a monovalent cation, to a self-assembling peptide solution. The specific conditions may include conditions related to a mouth of a subject.

The self-assembling peptide may be an amphiphilic self-assembling peptide. By “amphiphilic” it is meant that the peptide comprises hydrophobic portions and hydrophilic portions. In some embodiments, an amphiphilic peptide may comprise, consist essentially of, or consist of alternating hydrophobic amino acids and hydrophilic amino acids. By alternating, it is meant to include a series of three or more amino acids that alternate between a hydrophobic amino acid and a hydrophilic amino acid, and it need not include each and every amino acid in the peptide sequence alternating between a hydrophobic and a hydrophilic amino acid. The self-assembling peptide, also referred to herein as “peptide” may be administered to the pre-determined or desired target area in the form of a self-assembling peptide solution, composition, hydrogel, membrane, scaffold or other form. The hydrogel may also be referred to as a membrane or scaffold throughout this disclosure. The predetermined or desired target area may be located in an alveolar bone of a subject, such as in the posterior maxilla. The predetermined or desired target area may be established so as to facilitate a sinus lift procedure.

The self-assembling peptide solution may be an aqueous self-assembling peptide solution. The self-assembling peptide may be administered, applied, or injected in a solution that is substantially cell-free, or free of cells. In certain embodiments, the self-assembling peptide may be administered, applied, or injected in a solution that is cell-free or free of cells. The self-assembling peptide may also be administered, applied, or injected in a solution that is substantially drug-free or free of drugs. In certain embodiments, the self-assembling peptide may be administered, applied, or injected in a solution that is drug-free or free of drugs. In certain other embodiments, the self-assembling peptide may be administered, applied, or injected in a solution that is substantially cell-free and substantially drug-free. In still further certain other embodiments, the self-assembling peptide may be administered, applied, or injected in a solution that is cell-free and drug free.

The self-assembling peptide solution may comprise, consist of, or consist essentially of the self-assembling peptide. The self-assembling peptide may be in a modified or unmodified form. By modified, it is meant that the self-assembling peptide may have one or more domains that comprise one or more amino acids that, when provided in solution by itself, would not self-assemble. By unmodified, it is meant that the self-assembling peptide may not have any other domains other than those that provide for self-assembly of the peptide. That is, an unmodified peptide consists of alternating hydrophobic and hydrophilic amino acids that may self-assemble into a beta-sheet, and a macroscopic structure, such as a hydrogel.

Administration of a solution may comprise, consist of, or consist essentially of administration of a solution comprising, consisting of, or consisting essentially of a self-assembling peptide comprising, consisting of, or consisting essentially of between about 7 amino acids and about 32 amino acids. Other peptides that do not comprise, consist of, or consist essentially of between about 7 amino acids and about 32 amino acids may be contemplated by this disclosure.

By alternating, it is meant to include a series of three or more amino acids that alternate between a hydrophobic amino acid and a hydrophilic amino acid, and it need not include each and every amino acid in the peptide sequence alternating between a hydrophobic and a hydrophilic amino acid.

The materials and methods may comprise administering a self-assembling peptide to a predetermined or desired target. The peptide may be administered as a hydrogel or form a hydrogel upon administration. A hydrogel is a term that may refer to a colloidal gel that is dispersed in water. The hydrogel may also be referred to as a membrane or scaffold throughout this disclosure. The systems and methods may also comprise applying a self-assembling peptide to a predetermined or desired target as a solution such as an aqueous peptide solution.

The term “administering,” is intended to include, but is not limited to, applying, introducing, or injecting the self-assembling peptide, in one or more of various forms including, but not limited to, by itself, by way of solution, such as an aqueous solution, or by way of a composition, hydrogel, or scaffold, with or without additional components.

The method may comprise introducing a delivery device at or near a predetermined or desired target area of a subject. The method may comprise introducing a delivery device comprising at least one of a syringe, pipette, tube, catheter, syringe catheter, or other needle-based device to the predetermined or desired target area of a subject. The self-assembling peptide may be administered by way of a syringe, pipette, tube, catheter, syringe catheter, or other needle-based device to the predetermined or desired target area of a subject. The gauge of the syringe needle may be selected to provide an adequate flow of a composition, a solution, a hydrogel, or a liquid from the syringe to the target area. This may be based in some embodiments on at least one of the amount of self-assembling peptide in a composition, peptide solution, or a hydrogel being administered, the concentration of the peptide solution, in the composition, or the hydrogel, and the viscosity of the peptide solution, composition, or hydrogel. The delivery device may be a conventional device or designed to accomplish at least one of to reach a specific target area, achieve a specific dosing regime, deliver a specific target volume, amount, or concentration, and deliver accurately to a target area.

The disclosed methods of filling a dental bone void may comprise introducing a delivery device into the mouth of the subject and positioning an end of the delivery device proximate the target site. Selective administration of the peptide may allow for enhanced and more targeted delivery of the peptide solution, composition, or hydrogel such that bone augmentation is successful and positioned in the desired location in an accurate manner. The selective administration may provide enhanced, targeted delivery that markedly improves the positioning and effectiveness of the treatment over conventional delivery devices. Delivery devices that may be used in the systems, methods, and kits of the disclosure may include a syringe, pipette, tube, catheter, syringe catheter, other needle-based device, tube or catheter.

Use of the delivery device may include use of accompanying devices, such as a guidewire used to guide the device into position, or an endoscope that may allow proper placement and visualization of the target area, and/or the path to the target area. The endoscope may be a tube that may comprise at least one of a light and a camera or other visualization device to allow images of the subject's body to be viewed.

The use of the delivery device, such as a syringe, pipette, tube, catheter, syringe catheter, other needle-based device, catheter, or endoscope may require determining the diameter or size of the opening in which there is a target area, such that at least a portion of the syringe, pipette, tube, syringe catheter, other needle-type device, catheter, or endoscope may enter the opening to administer the peptide, peptide solution, composition, or hydrogel to the target area.

In certain embodiments, the hydrogel may be formed in vitro and administered to the desired location in vivo. In certain examples, this location may be the area in which it is desired to promote bone growth. In other examples, this location may be upstream, downstream of the area, or substantially near the area. It may be desired to allow a migration of the hydrogel to the area in which it is desired to promote bone growth. Alternatively, another procedure may position the hydrogel in the area in which it is desired. The desired location or target area may be at least a portion of an area associated with a surgical procedure, such as a sinus lift procedure.

In certain aspects of the disclosure, the hydrogel may be formed in vivo. A solution comprising the self-assembling peptide, such as an aqueous solution, may be inserted to an in vivo location or area of a subject to prevent or reduce an obstruction or prevent or reduce a stenosis at that location. In certain examples, the hydrogel may be formed in vivo at one location, and allowed to migrate to the area in which it is desired to promote bone growth. Alternatively, another procedure may place the hydrogel in the area in which it is desired to promote bone growth. The peptides of the present disclosure may be in the form of a powder, a solution, a gel, or the like. Since the self-assembling peptide gels in response to changes in solution pH and salt concentration, it can be distributed as a liquid that gels upon contact with a subject during application or administration.

In certain environments, the peptide solution may be a weak hydrogel and, as a result, it may be administered by way of a delivery device as described herein.

In accordance with one or more embodiments, self-assembling peptides may promote bone growth, such as alveolar bone growth. In certain embodiments, this may be because the hydrogel, once in place, provides a scaffold to allow for an infiltration of cells that promote bone growth of the target area.

In accordance with one or more embodiments, a macroscopic scaffold is provided. The macroscopic scaffold may comprise, consist essentially of, or consist of a plurality of self-assembling peptides, each of which comprises, consists essentially of, or consists of between about 7 amino acids and about 32 amino acids in an effective amount that is capable of being positioned within a dental bone void to promote bone growth therein.

In accordance with some embodiments, the self-assembling peptides may be amphiphilic, alternating between hydrophobic amino acids and hydrophilic amino acids. In accordance with one or more embodiments, a subject may be evaluated to determine a need for dental bone augmentation. Once the evaluation has been completed, a peptide solution to administer to the subject may be prepared.

In some embodiments, a biologically active agent may be used with the materials and methods of the present disclosure. A biologically active agent may comprise a compound, including a peptide, DNA sequence, chemical compound, or inorganic or organic compound that may impart some activity, regulation, modulation, or adjustment of a condition or other activity in a subject or in a laboratory setting. The biologically active agent may interact with another component to provide such activity. The biologically active agent may be referred to as a drug in accordance with some embodiments herein. In certain embodiments, one or more biologically active agents may be gradually released to the outside of the peptide system. For example, the one or more biologically active agents may be gradually released from the hydrogel. Both in vitro and in vivo testing has demonstrated this gradual release of a biologically active agent. The biologically active agent may be added to the peptide solution prior to administering to a subject, or may be administered separately from the solution to the subject.

This disclosure relates to aqueous solutions, hydrogels, scaffolds, and membranes comprising self-assembling peptides, sometimes referred to as self-assembling oligopeptides. The peptides may be comprised of a peptide having about 6 to about 200 amino acid residues. The self-assembling peptides may exhibit a beta-sheet structure in aqueous solution in the presence of physiological pH and/or a cation, such as a monovalent cation, or other conditions applicable to the mouth of a subject. The peptides may be amphiphilic and alternate between a hydrophobic amino acid and a hydrophilic amino acid. In certain embodiments, the peptide may comprise a first portion that may be amphiphilic, alternating between a hydrophobic amino acid and a hydrophilic amino acid, and another portion or region that is not amphiphilic.

The peptides may be generally stable in aqueous solutions and self-assemble into large, macroscopic structures, scaffolds, or matrices when exposed to physiological conditions, neutral pH, or physiological levels of salt. Once the hydrogel is formed it may not decompose, or may decompose or biodegrade after a period of time. The rate of decomposition may be based at least in part on at least one of the amino acid sequence and conditions of its surroundings.

By “macroscopic” it is meant as having dimensions large enough to be visible under magnification of 10-fold or less. In preferred embodiments, a macroscopic structure is visible to the naked eye. A macroscopic structure may be transparent and may be two-dimensional, or three-dimensional. Typically each dimension is at least 10 μm, in size. In certain embodiments, at least two dimensions are at least 100 μm, or at least 1000 μm in size. Frequently at least two dimensions are at least 140 mm in size, 10-100 mm in size, or more.

In certain embodiments, the size of the filaments may be about 10 nanometers (nm) to about 20 nm. The interfilament distance may be about 50 nm to about 80 nm.

“Physiological conditions” may occur in nature for a particular organism, cell system, or subject which may be in contrast to artificial laboratory conditions. The conditions may comprise one or more properties such as one or more particular properties or one or more ranges of properties. For example, the physiological conditions may include a temperature or range of temperatures, a pH or range of pH's, a pressure or range of pressures, and one or more concentrations of particular compounds, salts, and other components. For example, in some examples, the physiological conditions may include a temperature in a range of about 20 to about 40 degrees Celsius. In some examples, the atmospheric pressure may be about 1 atm. The pH may be in the range of a neutral pH. For example, the pH may be in a range of about 6 to about 8. The physiological conditions may include cations such as monovalent metal cations that may induce membrane or hydrogel formation. These may include sodium chloride (NaCl). The physiological conditions may also include a glucose concentration, sucrose concentration, or other sugar concentration, of between about 1 mM and about 20 mM. The physiological conditions may include the local conditions of the mouth including sinus regions in some specific embodiments.

In some embodiments, the self-assembling peptides may be peptides of between about 6 amino acids and about 200 amino acids. In certain embodiments, the self-assembling peptides may be peptides of at least about 7 amino acids. In certain embodiments, the self-assembling peptides may be peptides of between about 7 amino acids and about 32 amino acids. In certain further embodiments, the self-assembling peptides may be peptides of between about 7 amino acids and about 17 amino acids. In certain other examples, the self-assembling peptides may be peptides of at least 8 amino acids, at least about 12 amino acids, or at least about 16 amino acids.

The peptides may also be complementary and structurally compatible. Complementary refers to the ability of the peptides to interact through ionized pairs and/or hydrogen bonds which form between their hydrophilic side-chains, and structurally compatible refers to the ability of complementary peptides to maintain a constant distance between their peptide backbones. Peptides having these properties participate in intermolecular interactions which result in the formation and stabilization of beta-sheets at the secondary structure level and interwoven filaments at the tertiary structure level.

Both homogeneous and heterogeneous mixtures of peptides characterized by the above-mentioned properties may form stable macroscopic membranes, filaments, and hydrogels. Peptides which are self-complementary and self-compatible may form membranes, filaments, and hydrogels in a homogeneous mixture. Heterogeneous peptides, including those which cannot form membranes, filaments, and hydrogels in homogeneous solutions, which are complementary and/or structurally compatible with each other may also self-assemble into macroscopic membranes, filaments, and hydrogels.

The membranes, filaments, and hydrogels may be non-cytotoxic. The hydrogels of the present disclosure may be digested and metabolized in a subject. The hydrogels may be biodegraded in 30 days or less. They have a simple composition, are permeable, and are easy and relatively inexpensive to produce in large quantities. The membranes and filaments, hydrogels or scaffolds may also be produced and stored in a sterile condition. The optimal lengths for membrane formation may vary with at least one of the amino acid composition, solution conditions, and conditions at the target site.

In certain embodiments, a method of performing a sinus lift in a subject is provided. The method may comprise introducing a delivery device proximate a target site in a posterior maxilla of a subject where promotion of alveolar bone growth is desired. The method may further comprise administering through the delivery device a solution comprising a self-assembling peptide comprising between about 7 amino acids and about 32 amino acids in an effective amount and in an effective concentration to form a hydrogel scaffold under physiological conditions to promote alveolar bone growth at the target site. The method may further comprise removing the delivery device from the mouth of the subject.

The method may further comprise visualizing a region or target area comprising at least a portion of the mouth. Visualizing the region or target area may comprise visualizing the region or target area during at least one of identifying the target area, introducing the delivery device, positioning the end of the delivery device in the target area, administering the solution, removing the delivery device, and monitoring the target site thereafter. Visualizing the region or target area may provide for selective administration of the solution. Visualizing may occur at any time before, during, and after the administration of the solution. Visualization may occur, for example, at a time period of at least one of about one week subsequent to administration, about four weeks subsequent to administration and about eight weeks subsequent to administration.

The solution to be administered may consist essentially of, or consist of, a self-assembling peptide comprising at least about 7 amino acids. The solution to be administered may consist essentially of, or consist of, a self-assembling peptide comprising between about 7 amino acids and about 32 amino acids. The peptide may be amphiphilic and at least a portion of the peptide may alternate between a hydrophobic amino acid and a hydrophilic amino acid.

Methods of facilitating embodiments of the present disclosure may comprise providing instructions for administering through a delivery device a solution comprising a self-assembling peptide comprising between about 7 amino acids and about 32 amino acids in an effective amount and in an effective concentration to form a hydrogel under physiological conditions to promote alveolar bone growth. The peptide may be amphiphilic and at least a portion of the peptide may alternate between a hydrophobic amino acid and a hydrophilic amino acid.

The methods of facilitating may comprise providing the solution comprising a self-assembling peptide comprising between about 7 amino acids and about 32 amino acids in an effective amount and in an effective concentration to form a hydrogel under physiological conditions to promote alveolar bone growth. The peptide may be amphiphilic and at least a portion of the peptide may alternate between a hydrophobic amino acid and a hydrophilic amino acid.

The methods of facilitating may comprise providing instructions to visualize a region or target area comprising at least a portion of the mouth and/or sinus region. The method may comprise providing instructions to visualize the target area or region during at least one of identifying the target area, introducing a delivery device, positioning an end of the delivery device in the target area, administering the solution, removing the delivery device, and monitoring thereafter. The method may comprise providing instructions to visualize the target area in a time period about one week, about four weeks, or about eight weeks subsequent to the administration. Instructions may be provided to monitor the area at the target area or surrounding the target area. Instructions may be provided to use the methods of the present disclosure during a surgical procedure, such as during a sinus lift procedure.

The self-assembling peptides may be composed of about 6 to about 200 amino acid residues. In certain embodiments, about 7 to about 32 residues may be used in the self-assembling peptides, while in other embodiments self-assembling peptides may have about 7 to about 17 residues. The peptides may have a length of about 5 nm.

The peptides of the present disclosure may include peptides having the repeating sequence of arginine, alanine, aspartic acid and alanine (Arg-Ala-Asp-Ala (RADA)), and such peptide sequences may be represented by (RADA)ₚ, wherein p=2-50, such as (RADA)₄ or RADA16 (i.e. RADARADARADARADA).

Each of the peptide sequences disclosed herein may provide for peptides comprising, consisting essentially of, and consisting of the amino acid sequences recited.

The present disclosure provides materials, methods, and kits for solutions, hydrogels, and scaffolds comprising, consisting essentially of, or consisting of the peptides recited herein.

A 1 weight per volume (w/v) percent aqueous (water) solution and a 2.5 w/v percent of (RADA)₄ is commercially available as the product PuraMatrix® peptide hydrogel offered by 3-D Matrix Co., Ltd.

Certain peptides may contain sequences which are similar to the cell attachment ligand RGD (Arginine-Glycine-Aspartic acid). The RAD-based peptides may be of particular interest because the similarity of this sequence to RGD. The RAD sequence is a high affinity ligand present in the extracellular matrix protein tenascin and is recognized by integrin receptors.

The self-assembly of the peptides may be attributable to hydrogen bonding and hydrophobic bonding between the peptide molecules by the amino acids composing the peptides.

The self-assembling peptides of the present disclosure may have a nanofiber diameter in a range of about 10 nm to about 20 nm and an average pore size is in a range of about 5 nm to about 200 nm. In certain embodiments, the nanofiber diameter, the pore size, and the nanofiber density may be controlled by at least one of the concentration of peptide solution used and the amount of peptide solution used, such as the volume of peptide solution. As such, at least one of a specific concentration of peptide in solution and a specific amount of peptide solution to provide at least one of a desired nanofiber diameter, pore size, and density to adequately provide for bone growth may be selected.

As used herein, an amount of a peptide, peptide solution or hydrogel effective to promote alveolar bone growth, an “effective amount” or a “therapeutically effective amount,” refers to an amount of the peptide, peptide solution or hydrogel, which is effective, upon single or multiple administration (application or injection) to a subject, in augmenting, treating, or in curing, alleviating, relieving or improving a subject with a bone void or other disorder beyond that expected in the absence of such treatment. This may include a particular concentration or range of concentrations of peptide in the peptide solution or hydrogel and additionally, or in the alternative, a particular volume or range of volumes of the peptide solution or hydrogel. The method of facilitating may comprise providing instructions to prepare at least one of the effective amount and the effective concentration.

The dosage, for example, volume or concentration, administered (for example, applied or injected) may vary depending upon the form of the peptide (for example, in a peptide solution, hydrogel, or in a dried form, such as a lyophilized form) and the route of administration utilized. The exact formulation, route of administration, volume, and concentration can be chosen in view of the subject's condition and in view of the particular target area or location that the peptide solution, hydrogel, or other form of peptide will be administered. Lower or higher doses than those recited herein may be used or required. Specific dosage and treatment regimens for any particular subject may depend upon a variety of factors, which may include the specific peptide or peptides employed, the dimension of the area that is being treated, the desired thickness of the resulting hydrogel that may be positioned in the desired target area, and the length of time of treatment. Other factors that may affect the specific dosage and treatment regimens include age, body weight, general health status, sex, time of administration, rate of degradation, the severity and course of the disease, condition or symptoms, and the judgment of the treating physician. In certain embodiments, the peptide solution may be administered in a single dose. In other embodiments, the peptide solution may be administered in more than one dose, or multiple doses. The peptide solution may be administered in at least two doses.

An effective amount and an effective concentration of the peptide solution may be selected to at least partially augment bone growth in a dental bone void such as during a sinus lift procedure. In some embodiments, at least one of the effective amount and the effective concentration may be based in part on a dimension or diameter of the target area and/or the amount of bone augmentation desired.

The effective amount may be, as described herein, an amount that may provide for an at least partial augmentation of alveolar bone, such as in the posterior maxilla of a patient. Various properties of the mouth and sinus region of the patient may contribute to the selection or determination of the effective amount including at least one of the dimension or diameter of the target area, the flow rate of one or more fluids at or near the target area, the pH at or near the target area, and the concentration of various salts at or near the target area. Additional properties that may determine the effective amount include various properties listed above, at various locations along a pathway in which the peptide solution is delivered.

The effective amount may include volumes of from about 0.1 milliliters (mL) to about 100 mL of a peptide solution. The effective amount may include volumes of from about 0.1 mL to about 10 mL of a peptide solution. The effective amount may include volumes of from about 1 mL to about 5 mL of a peptide solution. In certain embodiments, the effective amount may be about 0.5 mL. In other embodiments, the effective amount may be about 1.0 mL. In yet other embodiments, the effective amount may be about 1.5 mL. In still yet other embodiments, the effective amount may be about 2.0 mL. In some other embodiments, the effective amount may be about 3.0 mL. In certain embodiments, the effective amount may be approximately 0.1 mL to about 5 mL per 1 cm² of target area. In certain embodiments, the effective amount may be approximately 1 mL per 1 cm² of target area. This effective amount may be related to a concentration, such as a 2.5 weight per volume percent of a peptide solution of the present disclosure.

In some embodiments, a more effective bone augmentation may be achieved with a greater volume of peptide solution administered or a higher concentration of peptide in solution to be administered. This may allow a longer lasting or thicker hydrogel to form within the target area, allowing a more secure position of the hydrogel in the target area. It is possible that if a high enough volume is not selected, the hydrogel may not be effective at the target area for the desired period of time.

The effective concentration may be, as described herein, an amount that may provide for a desired level of bone augmentation. Various properties of the mouth and sinus region may contribute to the selection or determination of the effective concentration including at least one of a dimension or diameter of the target area.

The effective concentration may include peptide concentrations in the solution in a range of about 0.1 w/v percent to about 3.0 w/v percent. In certain embodiments, the effective concentration may be about 1 w/v percent. In other embodiments, the effective concentration may be about 2.5 w/v percent. In at least some embodiments, a stock solution of PuraMatrix® (1% w/v) may have a pH level of about 2.0 to about 3.0.

In certain embodiments, a peptide solution having a higher concentration of peptide may provide for a more effective hydrogel that has the ability to stay in place and provide effective bone growth. For purposes of delivering the peptide solution, higher concentrations of peptide solutions may become too viscous to allow for effective and selective administration of the solution. It is possible that if a high enough concentration is not selected, the hydrogel may not be effective at promoting bone growth at the target area for the desired period of time. The effective concentration may be selected to provide for a solution that may be administered by injection or other means using a particular diameter needle or other delivery device.

Methods of the disclosure contemplate single as well as multiple administrations of a therapeutically effective amount of the peptides, compositions, peptide solutions, membranes, filaments, and hydrogels as described herein. Peptides as described herein may be administered at regular intervals, depending on the nature, severity and extent of the subject's condition. In some embodiments, a peptide, composition, peptide solution, membrane, filament, or hydrogel may be administered in a single administration. In some embodiments, a peptide, composition, peptide solution, or hydrogel described herein is administered in multiple administrations. In some embodiments, a therapeutically effective amount of a peptide, composition, peptide solution, membrane, filament, or hydrogel may be administered periodically at regular intervals. The regular intervals selected may be based on any one or more of the initial peptide concentration of the solution administered, the amount administered, and the degradation rate of the hydrogel formed. For example, after an initial administration, a follow-on administration may occur after, for example, one week, two weeks, four weeks, six weeks, or eight weeks. The follow-on administration may comprise administration of a solution having the same concentration of peptide and volume as the initial administration, or may comprise administration of a solution of lesser or great concentration of peptide and volume. The selection of the appropriate follow-on administration of peptide solution may be based on imaging the target area and the area surrounding the target area and ascertaining the needs based on the condition of the subject. The predetermined intervals may be the same for each follow-on administration, or they may be different. This may be dependent on whether the hydrogel formed from the previous administration is partially or totally disrupted or degraded. The follow-on administration may comprise administration of a solution having the same concentration of peptide and volume as the initial administration, or may comprise administration of a solution of lesser or great concentration of peptide and volume. The selection of the appropriate follow-on administration of peptide solution may be based on imaging the target area and the area surrounding the target area and ascertaining the needs based on the condition of the subject.

The self-assembling peptides of the present disclosure, such as RADA16, may be peptide sequences that lack a distinct physiologically or biologically active motif or sequence, and therefore may not impair intrinsic cell function. Physiologically active motifs may control numerous intracellular phenomena such as transcription, and the presence of physiologically active motifs may lead to phosphorylation of intracytoplasmic or cell surface proteins by enzymes that recognize the motifs. When a physiologically active motif is present, transcription of proteins with various functions may be activated or suppressed. The self-assembling peptides of the present disclosure may lack such physiologically active motifs and therefore do not carry this risk. A sugar may be added to the self-assembling peptide solution to improve the osmotic pressure of the solution from hypotonicity to isotonicity, thereby allowing the biological safety to be increased. In certain examples, the sugar may be sucrose or glucose.

The optimal lengths for membrane formation may vary with the amino acid composition. A stabilization factor contemplated by the peptides of the present disclosure is that complementary peptides maintain a constant distance between the peptide backbones.

The peptides can be chemically synthesized or they can be purified from natural and recombinant sources. Using chemically synthesized peptides may allow the peptide solutions to be deficient in unidentified components such as unidentified components derived from the extracellular matrix of another animal. This property therefore may eliminate concerns of infection, including risk of viral infection compared to conventional tissue-derived biomaterials. This may eliminate concerns of infection including infections such as bovine spongiform encephalopathy (BSE), making the peptide highly safe for medical use.

The initial concentration of the peptide may be a factor in the size and thickness of the membrane, hydrogel, or scaffold formed. In general, the higher the peptide concentration, the higher the extent of membrane or hydrogel formation. Hydrogels, or scaffolds formed at higher initial peptide concentrations (about 10 mg/ml) (about 1.0 w/v percent) may be thicker and thus, likely to be stronger.

Formation of the membranes, hydrogels, or scaffolds may be very fast, on the order of a few minutes. The formation of the membranes or hydrogels may be irreversible. In certain embodiments, the formation may be reversible, and in other embodiments, the formation may be irreversible. The hydrogel may form instantaneously upon administration to a target area. The formation of the hydrogel may occur within about one to two minutes of administration. In other examples, the formation of the hydrogel may occur within about three to four minutes of administration. In certain embodiments the time it takes to form the hydrogel may be based at least in part on one or more of the concentration of the peptide solution, the volume of peptide solution applied, and the conditions at the area of application or injection (for example, the concentration of monovalent metal cations at the area of application, the pH of the area, and the presence of one or more fluids at or near the area). The process may be unaffected by pH of less than or equal to 12, and by temperature. The membranes or hydrogels may form at temperatures in the range of about 1 to 99 degrees Celsius.

The hydrogels may remain in position at the target area for a period of time sufficient to provide a desired effect using the methods and kits of the present disclosure. The desired effect may be to promote bone growth so as to at least partially fill a dental bone void, for example, as part of a sinus lift procedure.

The period of time that the membranes or hydrogels may remain at the desired area may be for one or more days, up to one or more weeks, and up to several months. In other examples, it may remain at the desired area for up to 30 days, or more. It may remain at the desired area indefinitely. In other examples, it may remain at the desired area for a longer period of time, until it is naturally degraded or intentionally removed. If the hydrogel naturally degrades over a period of time, subsequent application or injection of the hydrogel to the same or different location may be performed.

In certain embodiments, the self-assembling peptide may be prepared with one or more components that may provide for enhanced effectiveness of the self-assembling peptide or may provide another action, treatment, therapy, or otherwise interact with one or more components of the subject. For example, additional peptides comprising one or more biologically or physiologically active amino acid sequences or motifs may be included as one of the components along with the self-assembling peptide. Other components may include biologically active compounds such as a drug or other treatment that may provide some benefit to the subject. For example, an antibiotic may be administered with the self-assembling peptide, or may be administered separately.

The peptide, peptide solution, or hydrogel may comprise small molecular drugs to treat the subject or to prevent hemolysis, inflammation, and infection. The small molecular drugs may be selected from the group consisting of glucose, saccharose, purified saccharose, lactose, maltose, trehalose, destran, iodine, lysozyme chloride, dimethylisoprpylazulene, tretinoin tocoferil, povidone iodine, alprostadil alfadex, anise alcohol, isoamyl salicylate, α,α-dimethylphenylethyl alcohol, bacdanol, helional, sulfazin silver, bucladesine sodium, alprostadil alfadex, gentamycin sulfate, tetracycline hydrochloride, sodium fusidate, mupirocin calcium hydrate and isoamyl benzoate. Other small molecular drugs may be contemplated. Protein-based drugs may be included as a component to be administered, and may include erythropoietin, tissue type plasminogen activator, synthetic hemoglobin and insulin.

A component may be included to protect the peptide solution against rapid or immediate formation into a hydrogel. This may include an encapsulated delivery system that may degrade over time to allow a controlled time release of the peptide solution into the target area to form the hydrogel over a desired, predetermined period of time. Biodegradable, biocompatible polymers may be used, such as ethylene vinyl acetate, polyanhydrides, polyglycolic acid, collagen, polyorthoesters, and polylactic acid.

Any of the components described herein may be included in the peptide solution or may be administered separate from the peptide solution. Additionally, any of the methods and methods of facilitating provided herein may be performed by one or more parties.

A peptide, peptide solution, or hydrogel of the disclosure may be provided in a kit. Instructions for administering the solution to a target area of alveolar bone in a subject may also be provided in the kit. The peptide solution may comprise a self-assembling peptide comprising between about 7 and about 32 amino acids in an effective amount and in an effective concentration to form a hydrogel to promote bone growth. The instructions for administering the solution may comprise methods for administering the peptide, peptide solution, or hydrogel provided herein, for example, by a route of administration described herein, at a dose, volume or concentration, or administration schedule. The peptide may be amphiphilic and at least a portion of the peptide may alternate between a hydrophobic amino acid and a hydrophilic amino acid.

The kit may also comprise informational material. The informational material may be descriptive, instructional, marketing, or other material that relates to the methods described herein. In one embodiment, the informational material may include information about production of the peptide, peptide solution, or hydrogel disclosed herein, physical properties of the peptide, composition, peptide solution or hydrogel, concentration, volume, size, dimensions, date of expiration, and batch or production site.

The kit may also optionally include a device or materials to allow for administration of the peptide or peptide solution to the desired area. For example, a syringe, pipette, tube, catheter, syringe catheter, or other needle-based device may be included in the kit. Additionally, or alternatively, the kit may include a guidewire, endoscope, or other accompanying equipment to provide selective administration of the peptide solution to the target area.

The kit may comprise in addition to or in the alternative, other components or ingredients, such as components that may aid in positioning of the peptide solution, hydrogel or scaffold. Instructions may be provided in the kit to combine a sufficient quantity or volume of the peptide solution with a sucrose solution, that may or may not be provided with the kit. Instructions may be provided for diluting the peptide solution to administer an effective concentration of the solution to the target area. The instructions may describe diluting the peptide solution with a diluant or solvent. The diluant or solvent may be water. Instructions may further be provided for determining at least one of the effective concentration of the solution and the effective amount of the solution to the target area. This may be based on various parameters discussed herein, and may include the dimensions of the target area.

Other components or ingredients may be included in the kit, in the same or different compositions or containers than the peptide, peptide solutions, or hydrogel. The one or more components may include components that may provide for enhanced effectiveness of the self-assembling peptide or may provide another action, treatment, therapy, or otherwise interact with one or more components of the subject. For example, additional peptides comprising one or more biologically or physiologically active sequences or motifs may be included as one of the components along with the self-assembling peptide. Other components may include biologically active compounds such as a drug or other treatment that may provide some benefit to the subject. The peptide, peptide solution, or hydrogel may comprise small molecular drugs to treat the subject or to prevent hemolysis, inflammation, and infection, as disclosed herein. A sugar solution such as a sucrose solution may be provided with the kit. The sucrose solution may be a 20% sucrose solution. Other components which are disclosed herein may also be included in the kit.

In some embodiments, a component of the kit is stored in a sealed vial, for example, with a rubber or silicone closure (for example, a polybutadiene or polyisoprene closure). In some embodiments, a component of the kit is stored under inert conditions (for example, under nitrogen or another inert gas such as argon). In some embodiments, a component of the kit is stored under anhydrous conditions (for example, with a desiccant). In some embodiments, a component of the kit is stored in a light blocking container such as an amber vial.

As part of the kit or separate from a kit, syringes or pipettes may be pre-filled with a peptide, peptide solution, or hydrogel as disclosed herein. Methods to instruct a user to supply a self-assembling peptide solution to a syringe or pipette, with or without the use of other devices, and administering it to the target area through the syringe or pipette, with or without the use of other devices, is provided.

In accordance with one or more embodiments, a kit may include a syringe and a cannula to facilitate administration of the peptide solution. The kit may also include at least one wound dressing to facilitate healing and/or to hold the administered peptide solution in place. A bather configured to support a sinus membrane during a sinus lift procedure may be provided in the kit. One or more materials to be mixed with the peptide solution prior to or during administration may be provided, such as an antibiotic or an anti-inflammatory agent. Other materials may include an allograft or a ceramic material to be mixed with the peptide solution to promote bone growth. A dental implant may also be included in the kit.

In accordance with one or more embodiments, a kit may include a peptide hydrogel in an effective amount and an effective concentration based at least in part on a dimension of the target site. In some embodiments, the concentration effective to promote alveolar bone growth comprises a concentration in a range of about 0.1 w/v percent to about 3 w/v percent peptide. In at least some embodiments, the peptide hydrogel solution may be substantially non-biologically active. The peptide hydrogel solution may be substantially non-granular. In some embodiments, the self-assembling peptide in the kit comprises about 16 amino acids that alternate between a hydrophobic amino acid and a hydrophilic amino acid. In at least some embodiments, the kit includes Puramatrix® peptide hydrogel.

In accordance with one or more embodiments, the kit may include instructions to use the peptide hydrogel in a sinus lift procedure as discussed herein. The instructions may recite mixing an autograft or an allograft with the peptide solution prior to administration. In some embodiments, the instructions may be directed to a one-step procedure involving concurrent administration of the peptide solution and securing of an implant at the target site. In other embodiments, the instructions may be directed to a two-step procedure involving administration of the peptide solution at the target site and subsequent securing of an implant in augmented alveolar bone at the target site after a predetermined period of time. In some embodiments, the predetermined period of time is about three to about six months. In at least some embodiments, the instructions may direct a practitioner to provide additional doses of the peptide solution subsequent to initial administration and prior to implantation. The instructions may indicate that additional peptide solution may be administered at the time of implantation.

In some embodiments of the disclosure, the self-assembling peptides may be used as a coating on a device or an instrument. The self-assembling peptides may also be incorporated or secured to a support, such as gauze or a bandage, or a lining, that may provide a therapeutic effect to a subject, or that may be applied within a target area. The self-assembling peptides may also be soaked into a sponge for use.

In accordance with one or more embodiments, macroscopic structures can be useful for culturing cells and cell monolayers. Cells prefer to adhere to non-uniform, charged surfaces. The charged residues and conformation of the proteinaceous membranes promote cell adhesion and migration. The addition of growth factors, such as fibroblast growth factor, to the peptide macroscopic structure can further improve attachment, cell growth and neurite outgrowth. The porous macrostructure can also be useful for encapsulating cells. The pore size of the membrane can be large enough to allow the diffusion of cell products and nutrients. The cells are, generally, much larger than the pores and are, thus, contained.

In accordance with one or more embodiments, a macroscopic scaffold comprises a plurality of self-assembling peptides, wherein the self-assembling peptides self-assemble into a β-sheet macroscopic scaffold and wherein said macroscopic scaffold encapsulates living cells and wherein said cells are present in said macroscopic scaffold in a three-dimensional arrangement. One or more embodiments also encompass methods of regenerating a tissue comprising administering to a mammal a macroscopic scaffold comprising the disclosed self-assembling peptides at a target site. In at least some embodiments, periodontal tissue is regenerated such as during a sinus lift procedure. In additional embodiments, a scaffold for periodontal tissue regeneration comprises a self-assembling peptide described herein. As used herein in the context of tissue regeneration and/or periodontal tissue regeneration, a scaffold may be a degradable hydrogel.

The function and advantage of these and other embodiments of the methods and kits disclosed herein will be more fully understood from the example below. The following example is intended to illustrate the benefits of the disclosed treatment approach, but do not exemplify the full scope thereof.

### Example

### Single-Blind, Randomized, Controlled Feasibility Study of PuraMatrix® Bone Void Filler Versus Demineralized Freeze-Dried Bone Allograft (DFDBA) in Dental Alveolar Sinus Lift Procedures

### 1. Introduction

This testing was conducted to determine whether PuraMatrix® Bone Void Filler (BVF) (RADA16 in sterile water) can be used safely in the bone augmentation procedure known as sinus lift (maxillary sinus floor elevation) to prepare a site for dental implant placement. Safety and efficacy of use of PuraMatrix® for sinus lift procedures to prepare a site for dental implant placement was also determined.

It is provided that PuraMatrix® may be indicated as a general bone-void filler in intraoral defects, including sinus lift procedures. It was provided in sterile syringes, for single use.

The control product was demineralized freeze dried bone allograft (DFDBA) in granular form. The control was mixed with autogenous blood or saline for hydration and used according to the manufacturer's instructions.

### 2. Description of the Study

The study was a prospective single-center study, comparing the standard of care in sinus lift grafting (demineralized freeze dried bone allograft, or DFDBA) to the investigational product, PuraMatrix®. After screening, enrolled subjects were randomly assigned to treatment groups in a 2:1 ratio. The 2:1 ratio was selected to increase the exposure to the study device for safety evaluation.

Fifteen subjects were treated with graft material (10 PuraMatrix® and 5 control) in sinus elevation procedures. Six months after sinus elevation, at least one dental implant was placed and the resulting histologic bone core from the osteotomy site was preserved for histological analysis. Follow-up continued during the prosthesis placement and loading procedures. The final implant assessments were done six months after the implants were loaded, sixteen months after the graft.

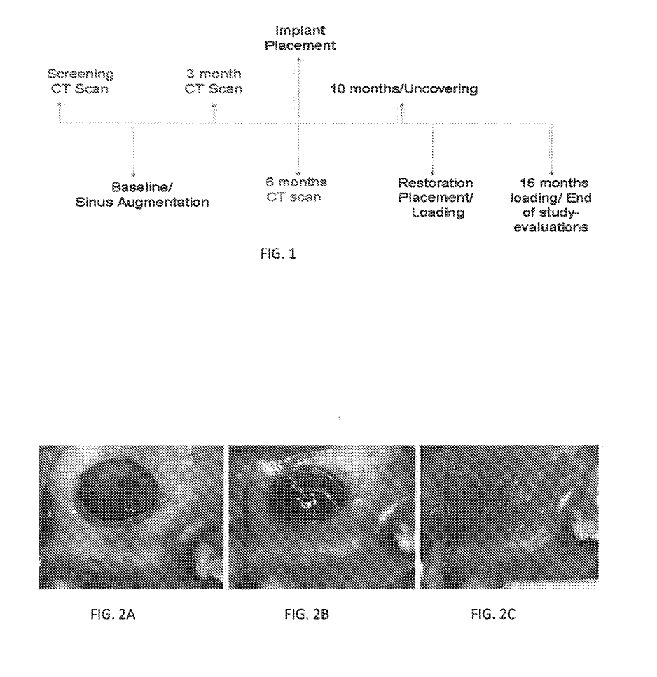

FIG. 1 is a schematic of the time line of the study protocol.

### 3. Study Objectives and Endpoints

The safety objective was to evaluate the safety of PuraMatrix® BVF in sinus lift procedures. The primary safety endpoint was the number and severity of implant-related control-related or procedure-related adverse events.

The efficacy objective was to evaluate the efficacy of PuraMatrix® BVF in bone regeneration in sinus lift procedures. The primary endpoints for efficacy were the qualitative evaluation of bone formed in the filled defect, assessed by both radiographic and histologic evaluations, and the quantitative measure of bone formation as evaluated by quantitative histomorphometry. The supplemental efficacy endpoint was implant success as defined using the Health Scale for Dental Implants, described further below in Table 2.

### 4. Endpoint Assessments

### Safety Assessment

Evaluation for humoral immune status was conducted by determining the level of serum IgG prior to treatment and at 3 months following bone graft treatment. If a subject had a result outside of the normal range at 3 months, an additional test was conducted at the 6 month follow up visit. Values out of range were not considered adverse events. Data on IgG levels is reported below in Table 1.

TABLE 1
Serum IgG Listing by Subject

| | | | Base- | | | | 3 | | | Date | Second | |
|---|---|---|---|---|---|---|---|---|---|---|---|---|
| | | | line | | | | month | | | second | graft | |
| | | Date of | IgG | Within | | Date of 3 | IgG | Within | Date of | graft | IgG | Within |
| Sub- | | baseline | value | normal | Date of | month | value | normal | second | sample | value | normal |
| ject | Group | sample | mg/dL | range? | graft | sample | mg/dL | range? | graft | taken | mg/dL | range? |
| 1 | DFDBA | Feb. 9, 2012 | 777 | Yes | Feb. 21, 2012 | May 21, 2012 | 746 | Yes | | | | Yes |
| 2 | PM | Feb. 23, 2012 | 818 | Yes | Mar. 6, 2012 | Jun. 4, 2012 | 874 | Yes | | | | Yes |
| 3 | PM | Feb. 27, 2012 | 1235 | Yes | Mar. 9, 2012 | Jun. 4, 2012 | 1234 | Yes | | | | Yes |
| 4 | PM | Mar. 12, 2012 | 1039 | Yes | Apr. 13, 2012 | Jul. 24, 2012 | 988 | Yes | Jan. 11, 2013 | Jan. 18, 2013 | 1100 | Yes |
| 5 | PM | Mar. 12, 2012 | 1419 | Yes | May 1, 2012 | Jul. 30, 2012 | 1309 | Yes | | | | Yes |
| 6 | DFDBA | Mar. 13, 2012 | 867 | Yes | Apr. 27, 2012 | Jul. 27, 2012 | 903 | Yes | | | | Yes |
| 7 | DFDBA | Mar. 26, 2012 | 1020 | Yes | Apr. 17, 2012 | Jul. 12, 2012 | 866 | Yes | | | | Yes |
| 8 | PM | Apr. 9, 2012 | 1177 | Yes | May 29, 2012 | Aug. 28, 2012 | 1044 | Yes | Dec. 12, 2012 | Dec. 19, 2012 | 1032 | Yes |
| 9 | PM | Apr. 9, 2012 | 1163 | Yes | Apr. 30, 2012 | Jul. 30, 2012 | 1177 | Yes | Jan. 15, 2013 | Feb. 20, 2013 | 1116 | Yes |
| 10 | PM | Apr. 11, 2012 | 1433 | Yes | May 11, 2012 | Aug. 21, 2012 | 1280 | Yes | | | | Yes |
| 12 | DFDBA | Apr. 24, 2012 | 1039 | Yes | May 25, 2012 | Aug. 28, 2012 | 955 | Yes | | | | Yes |
| 13 | PM | Apr. 24, 2012 | 1462 | Yes | May 15, 2012 | Aug. 20, 2012 | 1403 | Yes | | | | Yes |
| 14 | PM | Apr. 11, 2012 | 1166 | Yes | May 14, 2012 | Aug. 6, 2012 | 1129 | Yes | Jan. 10, 2013 | Jan. 17, 2013 | 1076 | Yes |
| 15 | DFDBA | May 3, 2012 | 838 | Yes | May 21, 2012 | Aug. 28, 2012 | 826 | Yes | | | | Yes |
| 16 | PM | Jun. 17, 2012 | 1077 | Yes | Jun. 14, 2012 | Sep. 17, 2012 | 1006 | Yes | | | | Yes |

### Efficacy Assessments

### Primary Efficacy

For measurement of primary efficacy outcomes, immediately after harvesting, each biopsy was marked on the crestal aspect and submerged in a 10% neutral buffered formalin solution for fixation. Following demineralization, cores were dehydrated and embedded in paraffin. Specimens were sectioned following a protocol accurately to obtain cylindrical sections at appropriate distances from the crestal portion of the sample. The cylindrical sections were sectioned parallel to the longitudinal axis according to conventional methods. Samples were stained with a conventional hematoxylin-eosin (H&E) technique and evaluated for histologic and histomorphometric analysis.

All samples were analyzed, using procedures and quantifications performed by a blinded histopathology technician. The analysis was performed using an optical microscope with an inverted digital camera. At least two slides of each height level per bone core specimen were analyzed. Images of the samples were captured at the same magnification. Quantification of the percent vital bone, remaining graft particle, and non-mineralized connective tissue were performed using specialized software (Image Pro-Plus Version 5.0). Vital bone was defined by the identification of osteocytes in the lacunae.

As an additional measure of efficacy, Cone Beam Computational Tomography (CBCT) scans were evaluated at the end of the study in a blinded fashion. Transverse sections of the sites were evaluated to measure the change in height and width of the alveolar bone between baseline and post augmentation. All measurements were made by one investigator.

### Secondary Efficacy Assessment

As a secondary outcome measure, implant success was evaluated by one investigator at prosthesis placement (four months after implant placement) and again at study completion using the ICOI Health Scale for Dental Implants, with success being defined as a score of II or better, as shown in Table 2.

TABLE 2
Health Scale for Dental Implants*

| Group | | Clinical Conditions | |
|---|---|---|---|
| I. | Success | a) | No pain or tenderness upon function |
| | (optimum | b) | No mobility |
| | health) | c) | <2 mm radiographic bone loss from initial |
| | | | surgery |
| | | d) | No exudates history |
| II. | Satisfactory | a) | No pain on function |
| | survival | b) | No mobility |
| | | c) | 2-4 mm radiographic bone loss |
| | | d) | No exudates history |
| III. | Compromised | a) | May have sensitivity on function |
| | survival | b) | No mobility |
| | | c) | Radiographic bone loss >4 mm (less |
| | | | than ½ of implant body) |
| | | d) | Probing depth >7 mm |
| | | e) | May have exudates history |
| IV. | Failure | Any of following: | |
| | (clinical or | a) | Pain on function |
| | absolute | b) | Mobility |
| | failure) | c) | Radiographic bone loss >½ length of |
| | | | implant |
| | | d) | Uncontrolled exudates |
| | | e) | No longer in mouth |

*International Congress of Oral Implantologists, Pisa, Italy, Consensus Conference, 2007.

### 5. Study Population

Fifteen subjects were treated during the study. The age range was 30 to 73 years, with a mean of 51. The enrollment period was 13 weeks in duration.

The 15 subjects available for treatment were randomized (2:1) to PuraMatrix® and DFDBA groups as indicated in the protocol. Randomization resulted in assignment of seven of the eight female subjects to the PuraMatrix® group. The mean age of subjects assigned to the PuraMatrix® group was 49 years, with a range of 30 to 72 years. The mean age of subjects assigned to the DFDBA group was 59 years, with a range of 51 to 73 years.

### 6. Treatment Procedure

Sinus augmentation for subjects in the investigational group was performed as follows.

### Control Treatment (DFDBA)

The DFDBA was mixed with autogenous blood for hydration according to the manufacturer's instructions. A resorbable collagen membrane (such as CollaTape®) was placed against the sinus membrane before placement of the graft material if necessary. The DFDBA was used according to manufacturer's instructions.

### PuraMatrix® Treatment

PuraMatrix® BVF may be used from cold storage or allowed to attain room temperature. No mixing is required. A resorbable collagen membrane (such as CollaTape®) was placed against the sinus membrane before placement of the graft material if necessary.

A supracrestal incision was made slightly toward the palatal aspect of the edentulous alveolar crest. The incision was extended between the remaining teeth or from the remaining teeth to the tuberosity in cases of edentulous distal extension. A mesial or distal vertical releasing incision was drawn when necessary to gain appropriate access. A full thickness mucoperiosteal flap was elevated for visualization of the lateral wall of the maxillary sinus. Then, a window was delineated with a round diamond bur, using the CBCT images as a reference. Once exposed, careful elevation of the Schneiderian membrane was performed using sinus membrane elevators. Sinus membrane was elevated up to 14 mm from the crest to allow sufficient implant length. The bone window was hinged over to membrane which formed the new base of the sinus. The membrane was protected after its elevation with a flat, blunt-edged metal instrument.

As much grafting material as necessary was placed to obtain a minimum height of 14 to 16 mm from the alveolar crest, and to fill up completely to the borders of the lateral window.

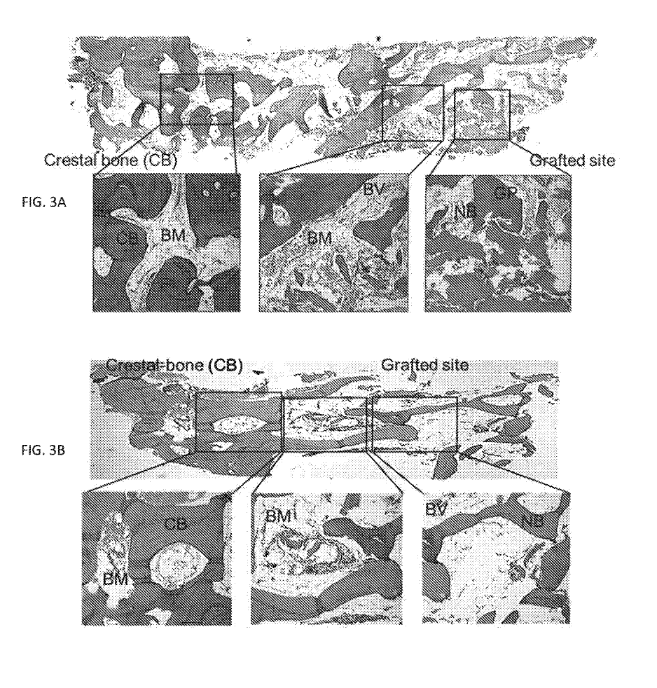

A representative case is shown in FIGS. 2A-2C (Lateral Wall Sinus Augmentation with PuraMatrix®).

Follow up visits included evaluation of wound healing and incidence of adverse events. Implant placement occurred at six months post graft. Multiple implants per subject were permitted, based on clinical judgment, and all subjects except two PuraMatrix® subjects with insufficient bone growth received at least one implant. Bone cores were harvested at the implant site and stored for histological evaluation. Bone level implants in diameters of 3.3, 4.1, or 4.8 were used (Straumann SLActive). A second stage surgery was performed to expose the implant fixture for prosthetic preparations. Prosthesis placement was performed per standard of care approximately four months after implant placement. The final follow up evaluation included implant stability rating and was conducted after the implant(s) had been loaded for 6 months.

### 7. Treatment Data

### Sinus Graft

The quantity of PuraMatrix® and DFDBA placed in the initial sinus lift grafting procedure is shown in Table 3. Note that less PuraMatrix® was placed than was DFDBA.

TABLE 3
Treatment Quantities (cc)

| | Initial | Second | |
|---|---|---|---|
| | treatment | treatment | Total |
| PuraMatrix ® | | | |
| N | 10 | 4 | 10 |
| Mean | 1.41 | 1.1 | 1.85 |
| Max | 2.6 | 1.5 | 4.1 |
| Min | 0.5 | 0.4 | 0.5 |
| Control | | | |
| N | 5 | 0 | 5 |
| Mean | 2.2 | | 2.2 |
| Max | 5.5 | | 5.5 |
| Min | 1.0 | | 1.0 |

It was observed that PuraMatrix® offers advantages over DFDBA in terms of handling and surgical technique. Considerably less time was necessary to prepare the graft. PuraMatrix® was found to be easy to apply, perfectly filling the surgical site, requiring less exposure time for the surgical site and therefore minimizing risk of contamination.

Six months after grafting, at implant placement, six PuraMatrix® subjects were found to have suboptimal bone quantity. The CBCT scans of two subjects showed bone height gain, but during the implant procedure the sites were found to be filled with fibrous tissue, not new bone. These two subjects were treated with the control product as the standard of care and exited the study. The remaining four subjects with limited bone growth were grafted with additional PuraMatrix® around the apical portion of the implant at placement to support the implants' long term stability. Primary stability was achieved on these implants and they continued in the study. An additional IgG test was performed on the subjects receiving an additional graft and no subject had values out of range. No related AEs were observed in these subjects.

### Implants and Abutments

Implants were placed in 12 of 17 graft locations in the PuraMatrix® group and all 6 locations in the DFDBA group. The designation of graft and implant placement locations is not precise, since it is based on tooth location terminology and the maxillary sinus spans multiple tooth locations. Therefore, discrepancies in location data are not considered significant, since the histologic evaluation of the bone cores confirmed that implants had been placed in grafted sites.

Primary stability at implant placement was confirmed for all placed implants in both groups and the torque measurement did not differ significantly between groups, ranging between 10 Ncm and 32 Ncm.

Abutments were placed and loaded after second stage surgery for each subject. At prosthesis placement, all implants were torqued to 35 Ncm to tighten the abutment screw and confirm that the implant had osseointegrated. A rating of Implant Quality was given at this visit as well. Implant quality at Prosthesis Placement was Successful in 12 and Satisfactory in 1 of the PuraMatrix® group and Successful in all 6 in the DFDBA group (Fisher exact p=1.000).

### IgG Results

All subjects, including those with two exposures to PuraMatrix®, showed serum IgG results within the normal range. A table of IgG values per subject is shown above in Table 1.

### Histopathology Results

The purpose of the histopathology portion of this study was to evaluate human bone core samples for bone formation and associated endpoints (i.e., quality of bone, remaining graft particles, % vital bone and % non-mineralized tissue) following lateral window sinus augmentation using PuraMatrix® or DFDBA bone void fillers at 6 months after placement.

In total, 7 DFDBA and 5 PuraMatrix® cores were utilized for histopathological evaluations. Two PuraMatrix® subjects were exited from the study after no bone growth was observed and 4 PuraMatrix® subjects did have sufficient bone to collect a core at implant placement for analysis. A full listing of bone cores taken is below in Table 4.

TABLE 4
Bone Cores Collected

| | | | Bone core | |
|---|---|---|---|---|
| | | | collected/ | |
| | | | implant | |
| Subject | Group | Tooth | placed? | Comments |
| 01-01 | DFDBA | 3 | Yes | |
| 01-02 | PuraMatrix | 13 | No | No bone growth, Implants |
| | | | | were not placed. Subject |
| | | | | removed from study. |
| | | 14 | No | No bone growth, Implants |
| | | | | were not placed. Subject |
| | | | | removed from study. |
| 01-03 | PuraMatrix | 3 | Yes | |
| | | 4 | Yes | Implant placed in site that |
| | | | | was not a grafted area, core |
| | | | | not analyzed |
| 01-04 | PuraMatrix | 14 | No | Insufficient bone |
| 01-05 | PuraMatrix | 3 | Yes | |
| | | 2 | Yes | Partial core only |
| 01-06 | DFDBA | 2 | Yes | |
| 01-07 | DFDBA | 3 | Yes | |
| 01-08 | PuraMatrix | 14 | No | Insufficient bone |
| | | 15 | No | Insufficient bone |
| 01-09 | PuraMatrix | 14 | No | Insufficient bone |
| 01-10 | PuraMatrix | 14 | Yes | |
| 01-12 | DFDBA | 12 | Yes | |
| | | 14 | Yes | |
| 01-13 | PuraMatrix | 14 | Yes | |
| | | 15 | Yes | Core dissolved, not |
| | | | | analyzed |
| 01-14 | PuraMatrix | 14 | No | Insufficient bone |
| 01-15 | DFDBA | 2 | Yes | |
| | | 3 | Yes | |
| 01-16 | PuraMatrix | 3 | No | No bone growth, Implants |
| | | | | were not placed. Subject |
| | | | | removed from study. |
| 01-16 | PuraMatrix | 4 | No | No bone growth, Implants |
| | | | | were not placed. Subject |
| | | | | removed from study. |

In general, all cores showed minimal inflammatory cell infiltration consistent with resorbing graft particles or material and normal bone turnover. No abscess formation was observed in any of the cores evaluated. DFDBA-grafted sites showed large resorbing graft particles surrounded by new bone, while PuraMatrix® cores showed greater new bone formation at the grafted sites.

The size of the bone marrow spaces was similar in both groups, supporting the findings with new bone in PuraMatrix® cores.

These results show that in this study, PuraMatrix® was capable of new bone formation in sinus augmentation procedures and that the results were comparable or superior to a standard treatment, DFDBA.

### Microscopic Observations

Both groups (PuraMatrix® and DFDBA) displayed varying degrees of inflammatory cell infiltration especially around graft particles (DFDBA) and graft material (PuraMatrix®) that indicate resorption of the graft material and bone turnover. No abscess formation was seen in any of the specimens. (See representative FIGS. 6-12). Overall, microscopic evaluations showed varying degrees of new bone formation at the grafted area in both groups. The crestal bone width varied between samples based on the initial crestal bone height (presence of <8 mm crestal bone for eligibility). The major difference between groups was the residual graft materials. They were present in all DFDBA-augmented sites, but the PuraMatrix® augmented sites showed minimal or almost no remaining graft material at 6 months post grafting. The grafted zone of the cores harvested from the DFDBA-augmented sites was mostly constructed by the residual graft materials with some connecting new bone bridges formed between the graft particles and the old bone (crestal bone). Bone marrow spaces were large but uniform for the entire specimen with blood vessels and non-mineralized tissue (see FIG. 3A). In PuraMatrix®-augmented site, the grafted zone mainly consisted of new bone matrix forming new trabecular structure with large and uniform marrow spaces with numberous blood vessels and non-mineralized tissue. Newly formed bone appeared to be more mature with osteocytes in lacunae compared to DFDBA grafted sites. Lining cells surrounded the newly formed bone indicating active new bone formation at the PuraMatrix® grafted sites while areas of dense inflammatory cell activity next to the newly formed bone was detected. This indicates the degradation of residual graft material and replacement by the newly formed bone as an active process (FIG. 3B).

FIG. 3A shows crestal zone of representative specimen at 100× magnification which had 6 mm of residual crest prior to grafting with DFDBA. The left end of the image shows crestal bone (CB) with mature bone elements and laminar organization while the right end shows new bone (NB) layered around graft particles (GP) with empty lacunae (depicted by arrow pointing to left). AT the grafted area, increased vascularization is detectable with large number of blood vessels (BV) and new bone formation around graft particles. New bone (NB) appears to be encapsulating graft particles (GP) with a cement line as the initial layer and a bridge between particles (Grafted site, depicted by arrows pointing to right). New bone formation was attached to graft particles.

FIG. 3B shows crestal zone of representative specimen at 100× magnification which had 5.3 mm of residual crest prior to grafting with PuraMatrix®. Similar to FIG. 3A, the left end of the image shows crestal bone (CB) with mature bone elements and laminar organization while the right end shows new bone (NB) with vital bone elements (osteocytes in lacunae (depicted by arrow pointing left)). Large marrow spaces (BM) with some blood vessels (BV) indicate more mature bone formation compared to DFDBA-grafted sites. Dense inflammatory cell infiltration was seen at the center of the core possibly surrounding the resorbing graft material. Note, the bone particles forming from the center of the active site with graft degradation (depicted by arrow pointing right).

### Quantitative Measure of Bone Formation

### Histomorphometry

The percent vital bone in all zones was quantified using software (Image Pro-Plus Version 5.0). Percentages were calculated based on the total area of the images at 100X. Vital bone was defined by identification of osteocytes in the lacunae. Measurements were made at three zones, crestal, mixed (both crestal and grafted) and the grafted sites on each image. The averages of 3 sections per core per zone were used to calculate the percent vital bone for each area on each core. Mean values for each area and for total core were calculated with standard deviation for both groups (DFDBA and PuraMatrix®).

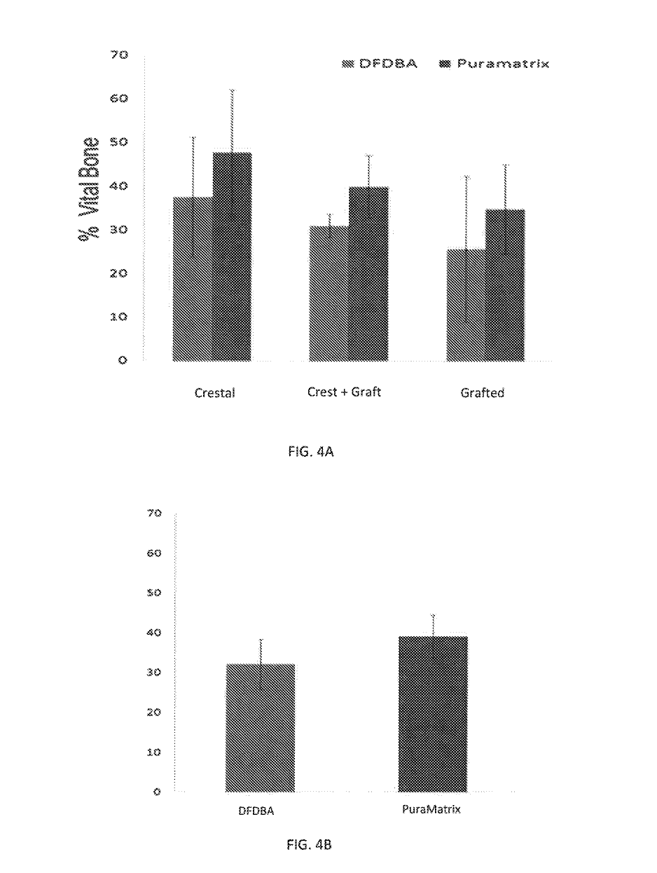

The percent vital bone in all zones (crestal mixed, and grafted zones) were greater in the cores augmented with PuraMatrix® than in those augmented with DFDBA (FIG. 4A). When total vital bone was calculated for each core, PuraMatrix®-grafted sites showed more vital bone compared to DFDBA-grafted sites (FIG. 4B). At the grafted zone of DFDBA sites, the bone structure was formed by mainly with graft particles surrounded by newly formed bone; while the grafted zone in PuraMatrix® cores were mainly formed by newly formed bone bridging to form trabecular structure of maxillary bone. Results are shown in Table 5: Histomorphometry Results.

### Percent Bone Marrow Space

The percent bone marrow space, which includes the non-mineralized connective tissue, fat tissue and bone vessels were quantified using software (Image Pro-Plus Version 5.0). Percentages were calculated based on the total area of the images at 100×. Measurements were made at three areas, cresta, mixed (both crestal and grafted) and the grafted sites on each image. The averages of 3 sections per core per area were used to calculate the % bone marrow space for each area on each core. Mean values for each area and for total core were calculated with standard deviation for both groups (DFDBA and PuraMatrix®).

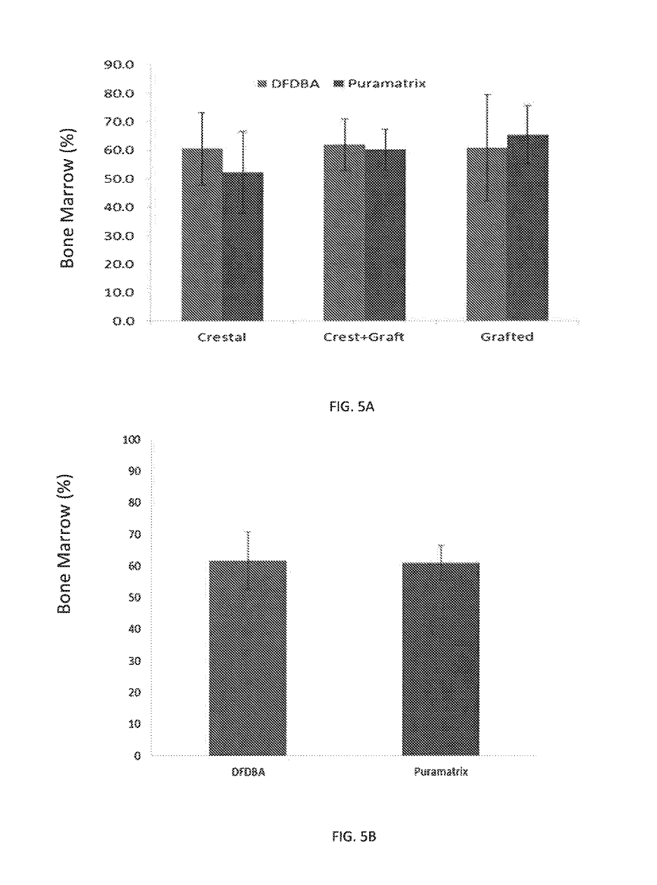

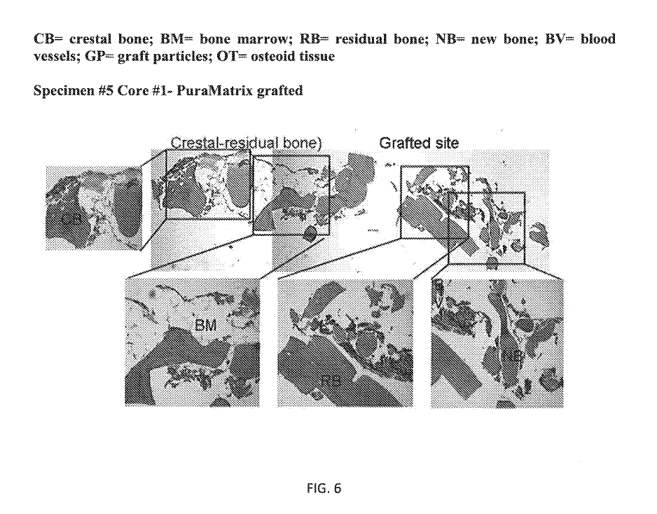

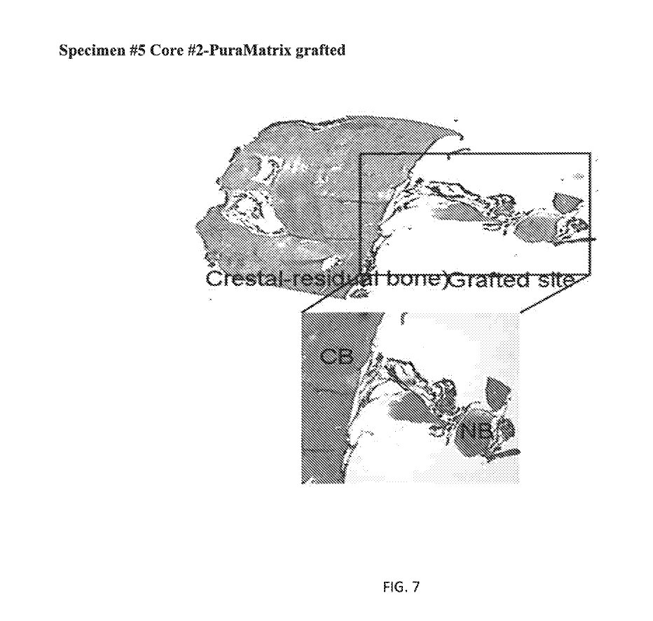

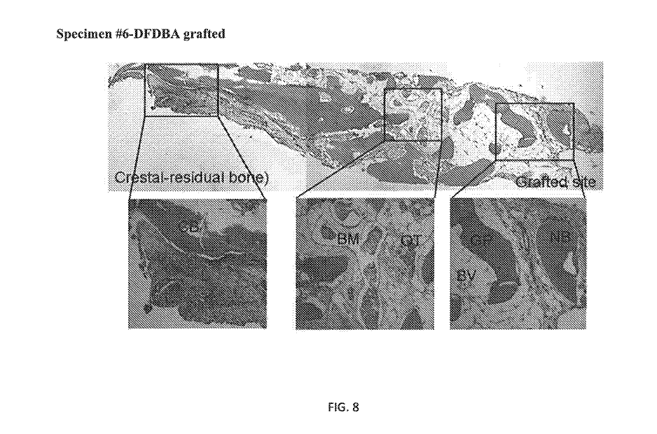

The percent bone marrow in all areas was similar in both groups compared to the difference in % vital bone. This was due to the non-resorbed graft particles that were largely seen in grafted areas of DFDBA cores. The results were similar in total bone marrow spaces when calculated for the entire core (FIG. 5, Table 5). There was no difference in total bone marrow space between groups, which also show that PuraMatrix® cores were formed by more new bone compared to control sites while DFDBA-grafted sites had a composite structure with residual graft particles and new bone surrounding the graft particles at the time of the bone core harvesting (6 months).

### Representative Images

Representative images are provided in FIGS. 6-13. FIG. 6 depicts a PuraMatrix® grafted area. Inflammatory cell infiltration can be seen around new bone indicating resorbing graft material. Lining cells surround the new mature bone with blood vessels in the bone marrow spaces. Thin residual bone is seen at the crestal area (˜0.5 mm) immediately adjacent to the grafted area with new bone activity.

FIG. 7 depicts a PuraMatrix® grafted area. The grafted site shows new forming bone both with active vasculature. Residual bone at the crestal level is seen with normal bone characteristics.

FIG. 8 depicts a DFDBA grafted area. The image shows minimal crestal residual bone with a core that is mostly formed by graft particles surrounded by new bone formation as thin layers. In the middle, non-mineralized tissue is observed. Large marrow spaces, some degree of vascularization and new bone formation are seen.

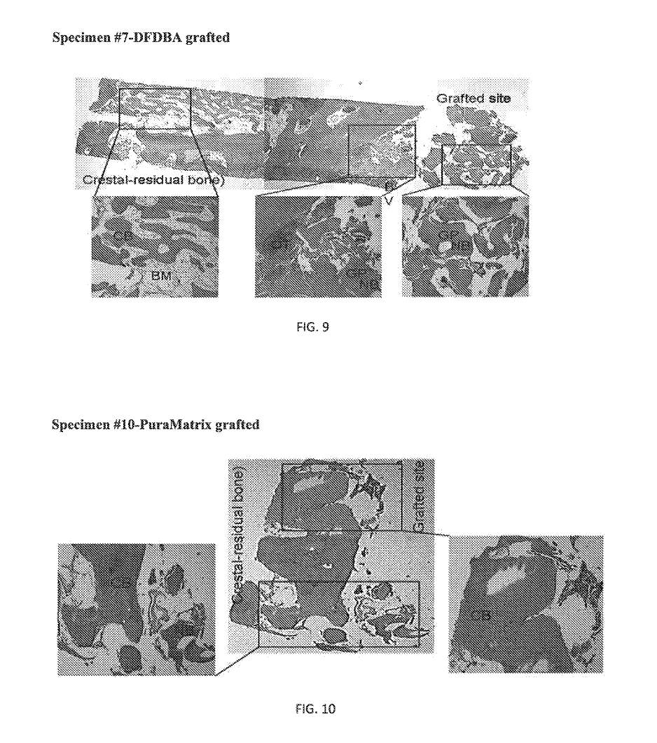

FIG. 9 depicts a DFDBA grafted area. A well distinguished non-mineralized tissue separating the residual crestal bone and grafted area is seen. Grafted area shows osteoid tissue between graft particles and surrounding new bone forming.

FIG. 10 depicts a PuraMatrix® grafted area. At the grafted area, new bone activity with inflammatory cells surrounding osteoid tissue formation is detected. New bone formation at the grafted site with large marrow spaces is observed. A thin residual crestal area with well organized mature bone is present.

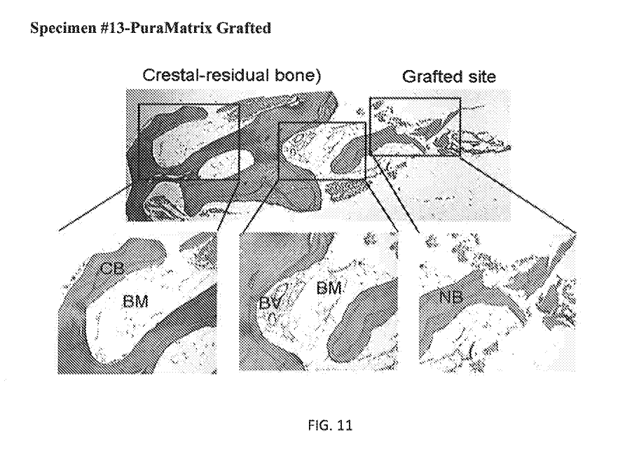

FIG. 11 depicts a PuraMatrix® grafted area. New bone particles with dense vascularization as well as inflammatory cell infiltration around new bone are seen at the grafted site. At the crestal area, well organized trabecular bone structure with large marrow spaces with less vascularization is present.

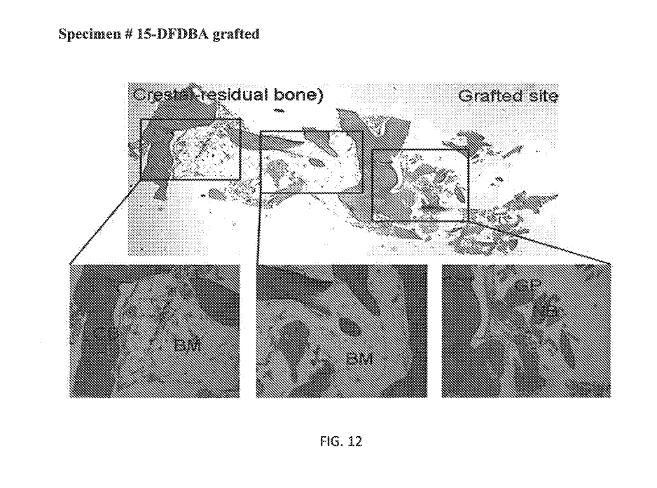

FIG. 12 depicts a DFDBA grafted area. The grafted area is mostly consisted of graft particles with newly formed bone and non-mineralized connective tissue surrounding the graft particles. Dense inflammatory cell infiltration around graft particles is present indicating bone turnover. Crestal area shows large bone marrow spaces with residual bone and less and smaller blood vessels indicating non-active mature bone.

### Radiography

The radiographic evaluations were performed to three-dimensionally evaluate alveolar bone height and width changes following lateral window sinus augmentation procedure using PuraMatrix® or DFDBA bone substitutes at three different time points: baseline, 3 months, and 6 months.

The radiographic evaluations were conducted on the images obtained by CBCT acquired using standard techniques according to the Operator's Manual (iCAT®, Imaging Sciences International). All procedures and quantifications were performed in blinded fashion by a single recorder.

At screening, a CBCT scan was taken from all enrolled subjects to determine eligibility for the study. This scan also served as a baseline measurement for bone height and width at the area of interest. Three (3) and 6 months following sinus augmentation with either PuraMatrix® or DFDBA bone substitute materials, the CBCT evaluations were repeated.

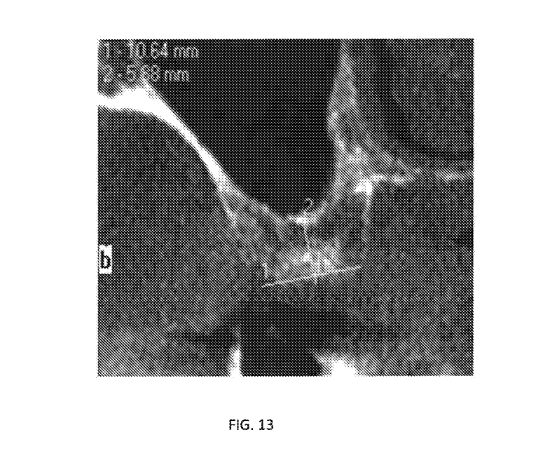

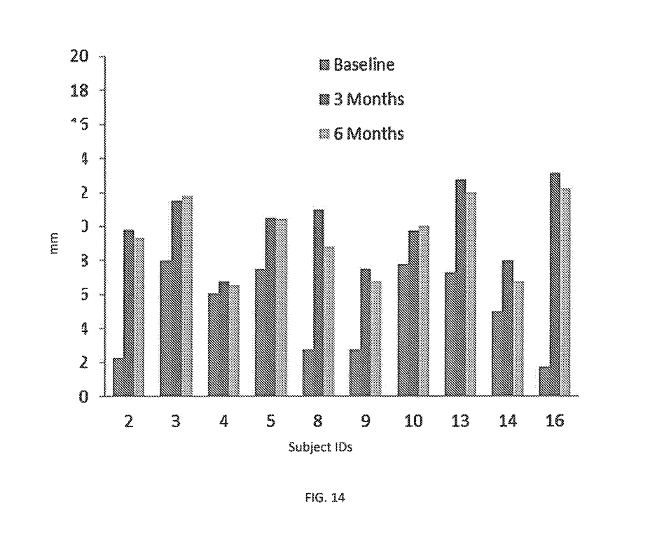

Alveolar bone height was measured as the distance between crestal bone edge and base of the sinus membrane while alveolar bone width was measured as the buccolingual dimension of the alveolar crest (FIG. 13). In FIG. 13, alveolar bone height is labeled as 2 and width is labeled as 1. They were measured using a “distance” tool of the CBCT software and the results presented in mm (e.g., 1=width=10.64 mm and 2=height=5.88 mm). These measurements were performed on three CBCT images, 1) prior to augmentation procedure, 2/at 3 months and 3) at 6 months. Each of the transverse sections used for measurements was selected from the site of interest (augmentation site) for each case and standardized by the selected region.

CBCT images showed significant changes in bone height for most of the subjects treated with PuraMatrix®, while in subjects #4, #9, #8, and #14, the change was limited, as shown in FIG. 14. Note that subjects #2 and #16 were found with insufficient bone formation and fibrous tissue formation in the augmentation site and excluded from the study at 6 months although the CBCT measurements showed significant bone height changes. In addition Subjects #4, #8, #9, and #14 received additional PuraMatrix® material at 6 months during implant placement due to limited bone formation. Overall, significant changes were observed at 3 and 6 months compared to baseline (p<0.05); with no difference between 3 and 6 months.

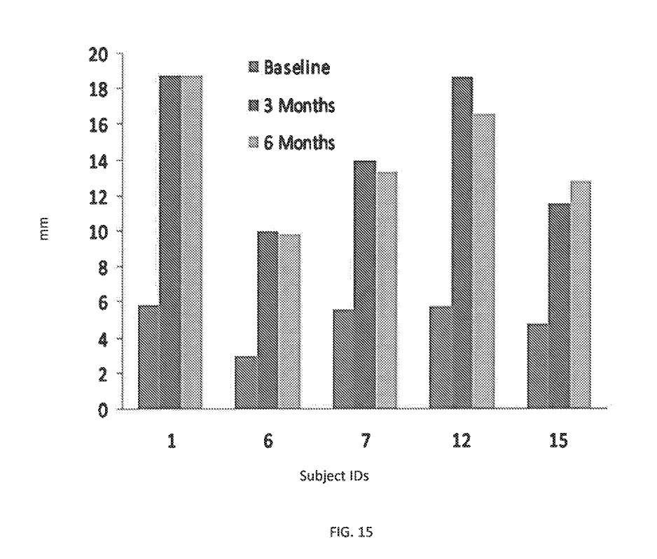

FIG. 15 shows changes in bone height in subjects assigned to DFDBA/Control group. Changes in 3 and 6 months were statistically significant compared to baseline (p<0.05); however, there was no statistically significant difference in bone height between 3 and 6 months.

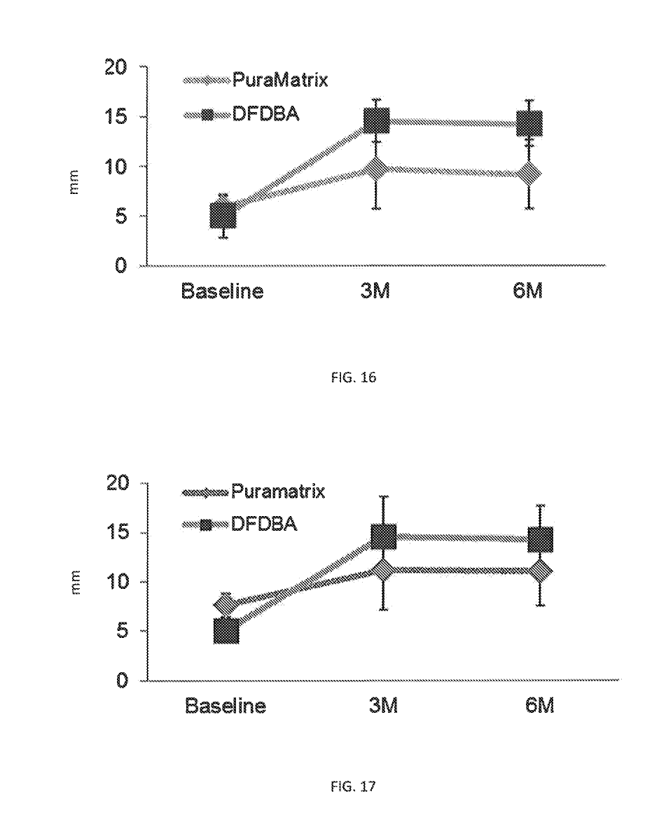

FIG. 16 shows a bone height comparison between PuraMatrix® and DFDBA groups (mean±SD) over time. Statistically significant differences in bone height were detected between PuraMatrix® and control groups at 3 and 6 months (*p=0.045 and *p=0.025, respectively). Note that subjects #2 and #16 in PuraMatrix® group were excluded from data analysis.

FIG. 17 shows a bone height change comparison between PuraMatrix® and DVDBA groups after excluding the failed and additionally grafted cases. The difference (mm) between groups at any time point was not statistically significant (p>0.05).

For both PuraMatrix® and DFDBA, augmentation resulted in significant increases in bone height at 3 months (p=0.002, p<0.0001, respectively) and at 6 months (p=0.009, p<0.001), relative to baseline. Overall bone height increase was greater for DFDBA than for PuraMatrix® (p=0.025). Bone height increase was more variable for PuraMatrix® than for DFDBA, based on the PuraMatrix® cases discussed previously, in which bone formation did not occur or was insufficient. When these cases were excluded from analysis, the differences in bone height between PuraMatrix® and DFDBA were not statistically significant.

Note that the volume of DFDBA used for grafting was, in general, more than the volume of PuraMatrix®. Another explanation for the somewhat lower gain in bone height for PuraMatrix®, relative to DFDBA, without wishing to be bound by theory, is that PuraMatrix® was not rigid enough to hold the sinus membrane at the position where elevated. This would be the case especially in large sinus cavities and with thick sinus membranes that tend to collapse during healing. To overcome this limitation, it is suggested that PuraMatrix® can be used in conjunction with a more rigid barrier as discussed herein against the sinus membrane in order to improve space maintenance for optimum new bone formation. With this configuration, PuraMatrix® would be able to fill its primary role as a scaffold for new bone formation, while being assisted by the membrane in the function of maintaining space.

Bone width did not show any significant changes over time in either group, indicating that the surgical procedure did not cause bone loss.

Tables 6 and 7 show the details of the measurements performed.

TABLE 7
Multiple computations of bone height changes between PuraMatrix ® and DFDBA
treated cases over time (ANOVA followed by Bonferroni test).

| | | Mean | | | 95% Confidence Interval | |
|---|---|---|---|---|---|---|
| (I) group time | (J) group time | Difference (I-J) | Std. Error | Sig. | Lower Bound | Upper Bound |
| PM-5 | PM-3M | −4.94300* | 1.15751 | .002 | −6.5624 | −1.3236 |
| | PM-5M | −4.32900* | 1.15751 | .009 | −7.9464 | −.7096 |
| | DFDBA-5 | −12500 | 1.41766 | 1.000 | −4.304 [symbol] | 4.5 [symbol] 08 |
| | DFDBA-3M | −9.43000* | 1.41766 | .000 | −13. [symbol] 62 [symbol] | −4.9972 |
| | DFDBA-5M | −9.12400* | 1.41766 | .000 | −13.5 [symbol] | −4. [symbol] 912 |
| PM-3M | PM-5 | 4.94300* | 1.15751 | .002 | 1.3236 | [symbol] .5624 |
| | PM-5M | . [symbol] 1400 | 1.15751 | 1.000 | −3.00 [symbol] 4 | 4.2334 |
| | DFDBA-5 | 5.07100* | 1.41766 | .014 | .6 [symbol] 82 | 9. [symbol] 038 |
| | DFDBA-3M | −4.4 [symbol] 700* | 1.41766 | .045 | −5.9196 | −0.542 |
| | DFDBA-5M | −4.18100 | 1.41766 | .060 | −5.6136 | .2518 |
| PM-6M | PM-5 | 4.32900* | 1.15751 | .009 | .7096 | 7.9464 |
| | PM-3M | −. [symbol] 1400 | 1.15751 | 1.000 | −4.2334 | 3.0054 |
| | DFDBA-5 | 4.45700* | 1.41766 | .048 | .0242 | [symbol] . [symbol] 898 |
| | DFDBA-3M | −5.10100* | 1.41766 | .013 | −9.533 [symbol] | . [symbol] 6 [symbol] 2 |
| | DFDBA-5M | −4.79500* | 1.41766 | .025 | −9.2276 | −.3622 |
| DFDBA-B | PM-5 | −.12500 | 1.41766 | 1.000 | −4.5608 | 4.3048 |
| | PM-3M | −5.07100* | 1.41766 | .014 | −9.5038 | −.6362 |
| | PM-6M | −4.45700* | 1.41766 | .046 | −. [symbol] . [symbol] 9 [symbol] | −.0242 |
| | DFDBA-3M | −9.55 [symbol] 00* | 1. [symbol] 97 | .000 | −14.67 [symbol] 6 | −4.4394 |
| | DFDBA-5M | −9.25200* | 1. [symbol] 97 | .000 | −14.3706 | −4.1334 |
| DFDBA-3M | PM-5 | 9.43000* | 1.41766 | .000 | 4.9972 | 13.6626 |
| | PM-3M | 4.4 [symbol] 700* | 1.41766 | .045 | .0542 | [symbol] .9198 |
| | PM-5M | 5.10100* | 1.41766 | .013 | .66 [symbol] 2 | 9.53338 |
| | DFDBA-5 | 9.55 [symbol] 00* | 1.53697 | .000 | 4.4394 | 14. [symbol] 7 [symbol] |
| | DFDBA-5M | .30600 | 4.53697 | 1.000 | −4. [symbol] 126 | 5.424 [symbol] |
| DFDBA-5M | PM-5 | 9.12400* | 1.41766 | .000 | 4.6912 | 13.56 [symbol] |
| | PM-3M | 4.18100 | 1.41766 | .080 | −.2518 | 8.6138 |
| | PM-5M | 4.79500* | 1.41765 | .025 | .3622 | 9.227 [symbol] |
| | DFDBA-5 | 9.25200* | 1. [symbol] 3597 | .000 | 4.1334 | 14.3706 |
| | DFDBA-3M | −.30600 | 1.63697 | 1.000 | −5.4246 | 4.8126 |

PM = PuraMatrix,
B = Baseline,
3 M = 3 months,
6 M = 6 months.
Significances are shown in [symbol] for both groups compared to baseline ane between groups. [symbol]
indicates data missing or illegible when filed

### Assessment of Implant Success Using Health Scale for Dental Implants Analysis

Implant success according to the Health Scale for Dental Implants at prosthesis placement (loading—10 months after graft placement) and at six months after prosthesis placement is summarized in Table 8 and Table 9 for PuraMatrix® and DFDBA, respectively. All implants in both groups were successful according to the predefined criteria. In addition to the clinical evaluation according to the Health Scale for Dental Implants, the fact that all implants survived application of 35 N-cm torque at prosthesis placement confirmed that they were osseointegrated. All implants had a rating of I at abutment placement and at 6 months after prosthesis placement, except for one implant in a site grafted with PuraMatrix®, which had a success rating of II at abutment placement. The same implant had a success rating of I at 6 months after prosthesis placement. This implant site had received additional PuraMatrix® at the time of implant placement.

TABLE 8
Implant success - PuraMatrix ®

| | | Success | Success |
|---|---|---|---|
| | Location | rating 10 | rating 16 |
| | of | months | months |
| Subject | implant | after graft | after graft |
| 3 | 3 | I | I |
| | 4 | I | I |
| 4* | 14 | II | I |
| 5 | 3 | I | I |
| | 2 | I | I |
| 8* | 14 | I | I |
| | 15 | I | I |
| 9* | 14 | I | I |
| 10 | 14 | I | I |
| 13 | 14 | I | I |
| | 15 | I | I |
| 14* | 14 | I | I |

*Subject received 2 doses of PuraMatrix ®

TABLE 9
Implant success - control

| | | Success | Success |
|---|---|---|---|
| | Location | rating 10 | rating 16 |
| | of | months | months |
| Subject | Implant | after graft | after graft |
| 1 | 3 | I | I |
| 6 | 2 | I | I |
| 7 | 3 | I | I |
| 12 | 12 | I | I |
| | 14 | I | I |
| 15 | 3 | I | I |
| | 2 | I | I |

### CONCLUSIONS

This study showed that PuraMatrix® can be safely and successfully used in sinus augmentation procedures. The safety objective was met by demonstrating that the number and severity of adverse events for PuraMatrix® is similar to that of the control treatment. The efficacy objective was met by showing that the formation of new vital bone is similar or superior to that observed for the control treatment. The supplemental efficacy objective was met by showing that, for PuraMatrix® and the control treatment, implants placed in the graft were successful at six months after prosthesis placement (loading), as defined by the Health Scale for Dental Implants.

It may be beneficial to use PuraMatrix® in conjunction with a more rigid barrier, in order to ensure space maintenance and optimum new bone formation.

Additionally, it was found that more time is necessary to prepare the graft (about 15 minutes, with dehydration and waiting time) when using DFDBA versus PuraMatrix®. It may be difficult to predict the exact amount needed, and therefore, it may take more time to prepare an additional graft, if needed. More time may also be needed with DFDBA to condense the graft in the augmented area. More care is also needed to carefully transfer the graft into the site. There may be more contamination risk or risk for loss of graft during the transfer.

With PuraMatrix®, it was found that considerably less time is necessary to prepare the graft (about 2-5 minutes, no dehydration or waiting time). It is easy to predict the exact amount needed, and if an additional amount is needed, it takes only one to two minutes to add a new syringe containing PuraMatrix®. There is almost no extra time needed to condense the graft in the augmented area. Additionally, less contamination risk and less care is needed to transfer the graft into the surgical site.

It was also found that PuraMatrix® is easy and quick to apply. Therefore there is less exposure time for the surgical site. It may perfectly fill the surgical site, and there is no contamination risk or risk of loss of material. There is also no post-operative problems or clinical evidence of any intra-oral or extra-oral pathology.

Various embodiments of the materials and methods discussed herein are not limited in their application to the details as set forth in the description or illustrated in the drawings.

One or more embodiments are capable of being practiced or carried out in various ways beyond those exemplarily presented herein.

What is claimed is:

1. A method of performing a sinus lift procedure on a subject, comprising:
introducing a delivery device into a mouth of the subject;
positioning an end of the delivery device proximate a target site in a posterior maxilla of the subject where promotion of alveolar bone growth is desired;
administering through the delivery device a solution comprising a self-assembling peptide comprising between about 7 and about 32 amino acids in an effective amount and in an effective concentration to form a hydrogel scaffold under physiological conditions to promote alveolar bone growth at the target site; and
removing the delivery device from the mouth of the subject.

2. The method of claim 1, further comprising securing an implant into augmented alveolar bone at the target site after a predetermined period of time.

3. The method of claim 2, wherein the predetermined period of time is between about three and about six months.

4. The method of claim 2, wherein the augmented alveolar bone is characterized by a vital bone density of at least about 35%.

5. The method of claim 2, further comprising administering a supplemental volume of the peptide solution at the target site concurrently with implantation.

6. The method of claim 1, further comprising securing an implant at the target site in the posterior maxilla concurrently with administration of the peptide solution.

7. The method of claim 1, further comprising visualizing the target site after a pre-determined period of time to assess alveolar bone augmentation.

8. The method of claim 1, wherein the concentration effective to promote alveolar bone growth comprises a concentration in a range of about 0.1 weight per volume (w/v) percent to about 3 w/v percent peptide.

9. The method of claim 8, wherein the peptide in the solution comprises (RADA)₄.

10. The method of claim 1, wherein a volume of the administered peptide solution is between about 1 mL and about 5 mL.

11. The method of claim 1, further comprising supporting a sinus membrane in a region of the target site to provide a space for formation of the hydrogel scaffold.

12. The method of claim 1, wherein the self-assembling peptide comprises about 16 amino acids that alternate between a hydrophobic amino acid and a hydrophilic amino acid.

13. The method of claim 1, wherein the peptide solution is substantially non-biologically active.

14. The method of claim 1, further comprising mixing the peptide solution with an autograft or an allograft prior to administration.

15. The method of claim 1, wherein the method is associated with less than about 2 mm of radiographic bone loss at the target site upon implantation.

16. The method of claim 1, wherein the method is associated with no IgG reaction.

17. The method of claim 1, wherein the method is used after a surgical procedure.

18. The method of claim 1, further comprising applying a wound dressing at the target site after administration of the peptide solution.

19. The method of claim 1, wherein the hydrogel scaffold comprises nanofibers having a diameter of about 10 nanometers to about 20 nanometers.

20. A kit for filling a dental bone void in a subject, comprising:
a solution comprising a self-assembling peptide comprising between about 7 amino acids and about 32 amino acids in an effective amount and in an effective concentration to form a hydrogel scaffold under physiological conditions to promote alveolar bone growth at a target site; and
instructions for administering the solution to the target site in an alveolar bone of the subject.

21. The kit of claim 20, wherein at least one of the effective amount and the effective concentration is based in part on a dimension of the target site.

22. The kit of claim 20, wherein the concentration effective to promote alveolar bone growth comprises a concentration in a range of about 0.1 w/v percent to about 3 w/v percent peptide.

23. The kit of claim 22, wherein the peptide in the solution comprises (RADA)₄.

24. The kit of claim 20, wherein the peptide solution is substantially non-biologically active.

25. The kit of claim 20, wherein the self-assembling peptide comprises about 16 amino acids that alternate between a hydrophobic amino acid and a hydrophilic amino acid.

26. The kit of claim 20, wherein the peptide solution comprises at least one of an antibiotic and an anti-inflammatory agent.

27. The kit of claim 20, wherein the instructions recite mixing an autograft or an allograft with the peptide solution prior to administration.

28. The kit of claim 20, further comprising a ceramic to be mixed with the peptide solution prior to administration.

29. The kit of claim 20, wherein the instructions are directed to a one-step procedure involving concurrent administration of the peptide solution and securing of an implant at the target site.

30. The kit of claim 20, wherein the instructions are directed to a two-step procedure involving administration of the peptide solution at the target site and subsequent securing of an implant in augmented alveolar bone at the target site after a predetermined period of time.

31. The kit of claim 30, wherein the predetermined period of time is about three to about six months.

32. The kit of claim 20, further comprising a barrier configured to support a sinus membrane during the sinus lift procedure.

33. The kit of claim 20, wherein the peptide solution is substantially non-granular.

34. The kit of claim 20, further comprising at least one of a syringe and a cannula to facilitate administration of the peptide solution.

35. The kit of claim 20, further comprising a wound dressing.

36. The kit of claim 20, further comprising an allograft.

37. The kit of claim 20, further comprising a dental implant.
